(19) United States
(12) Patent Application Publication
(10) Pub. No.: US 2026/0248414 A1
(43) Pub. Date: Aug. 27, 2026

(54) SYSTEM

(71) Applicant: SoftBank Group Corp., Tokyo (JP)

(72) Inventor: Katsuto MINATSUKI, Tokyo (JP)

(73) Assignee: SoftBank Group Corp., Tokyo (JP)

(21) Appl. No.: 19/536,239

(22) Filed: Feb. 11, 2026

(30) Foreign Application Priority Data
Feb. 21, 2025 (JP) 2025-027085

(51) Int. Cl. A61B 5/11; A61B 5/00; A61B 5/0205; A61B 5/021; A61B 5/024; A61B 5/16; G01P 15/18; G06V 10/36; G06V 10/82; G06V 40/20

(52) U.S. Cl. CPC A61B 5/1117; A61B 5/02055; A61B 5/1112; A61B 5/1122; A61B 5/165; A61B 5/7203; A61B 5/7267; A61B 5/7282; A61B 5/747; G01P 15/18; A61B 5/0075; A61B 5/021; A61B 5/024; A61B 5/6803; A61B 2503/08; A61B 2505/07; A61B 2560/0242; A61B 2562/0219; G06V 10/36; G06V 10/82; G06V 40/20

(57) ABSTRACT

The system according to the embodiment comprises an operation detection unit, an analysis unit, a notification unit, a fall detection unit, an instruction unit, and a health monitoring unit. The operation detection unit detects an operation. The analysis unit analyzes information detected by the operation detection unit. The notification unit provides a notification based on information analyzed by the analysis unit. The fall detection unit detects a fall. The analysis unit analyzes information detected by the fall detection unit. The instruction unit instructs emergency response based on information analyzed by the analysis unit. The health monitoring unit monitors health. The analysis unit analyzes information monitored by the health monitoring unit. The notification unit provides a notification based on information analyzed by the analysis unit.

### CROSS-REFERENCE TO RELATED APPLICATION

The present application claims priority to and incorporates by reference the entire contents of Japanese Patent Application No. 2025-027085 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, there has been a problem that it is difficult to effectively monitor the safety and health of elderly persons living alone and to respond quickly in emergencies.

### SUMMARY OF THE INVENTION

The system according to the embodiment comprises an operation detection unit, an analysis unit, a notification unit, a fall detection unit, an instruction unit, and a health monitoring unit. The operation detection unit detects an operation. The analysis unit analyzes information detected by the operation detection unit. The notification unit provides a notification based on information analyzed by the analysis unit. The fall detection unit detects a fall. The analysis unit analyzes information detected by the fall detection unit. The instruction unit instructs emergency response based on information analyzed by the analysis unit. The health monitoring unit monitors health. The analysis unit analyzes information monitored by the health monitoring unit. The notification unit provides a notification based on information analyzed by the analysis unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

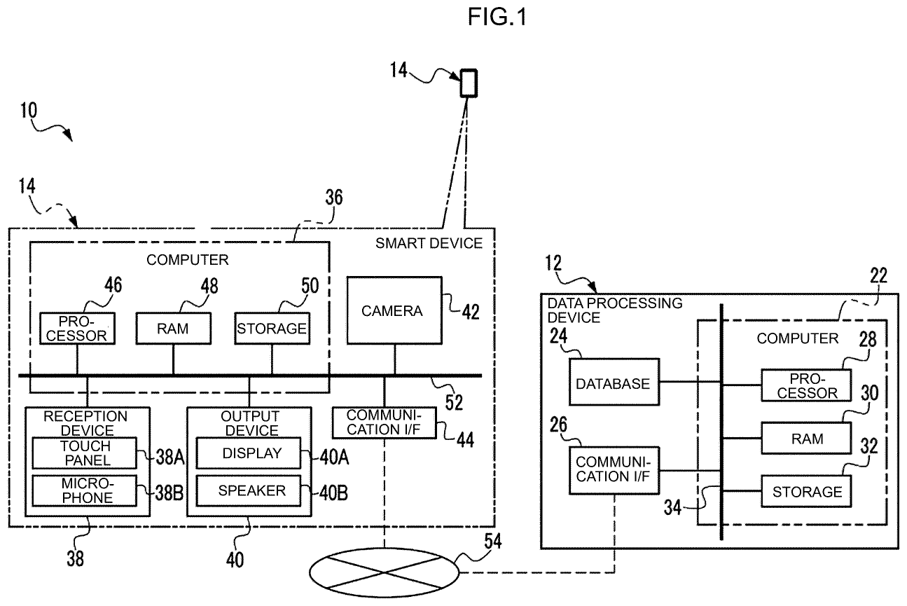

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

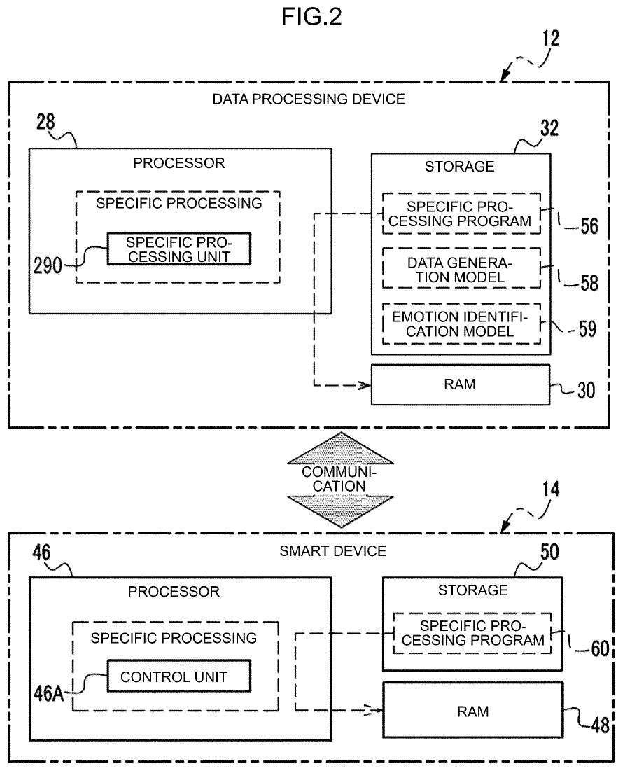

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The system according to the embodiment of the present invention is an AI-based solution that supports the safety and health of elderly persons living alone. This system combines motion sensors, fall detection devices, and medical monitoring systems to assist in ensuring the safety of elderly persons at home and providing emergency response through AI. Furthermore, by enhancing communication between users and family members or caregivers, it provides a safe and comfortable living environment. For example, the movement of an elderly person is detected using motion sensors. The motion sensors are installed in various locations in a room and monitor the movement of the elderly person in real time. For instance, the system detects the movement of the elderly person as they move around the room and checks for any abnormalities. If an abnormality is detected, AI analyzes the information and notifies family members or caregivers as necessary. Next, the fall of an elderly person is detected using a fall detection device. The fall detection device is worn on the body of the elderly person and immediately detects when a fall occurs. For example, if the elderly person falls, the device sends the information to AI, which instructs emergency response. This enables prompt response and ensures the safety of the elderly person. Furthermore, the health status of the elderly person is monitored using a medical monitoring system. The medical monitoring system measures vital signs such as body temperature, heart rate, and blood pressure of the elderly person in real time and sends the information to AI. AI analyzes these data and, if an abnormality is detected, notifies family members or caregivers. For example, if the body temperature of the elderly person rises rapidly, AI analyzes the information and notifies family members or caregivers, enabling prompt response. In addition, the system is equipped with functions to enhance communication between users and family members or caregivers. For example, there is a function for AI to periodically report the status of the elderly person to family members or caregivers, and a function that enables direct communication between family members or caregivers and the elderly person in an emergency. This allows the status of the elderly person to be constantly monitored, enabling them to live with peace of mind. Thus, the present invention combines motion sensors, fall detection devices, and medical monitoring systems to support the safety and emergency response of elderly persons at home through AI. Furthermore, by enhancing communication between users and family members or caregivers, it provides a safe and comfortable living environment. As a result, the system that supports the safety and health of elderly persons living alone can assist in ensuring safety and emergency response at home. Specifically, this system aggregates high-dimensional data collected from multiple sensor devices (e.g., three-axis acceleration vectors, time-series tensors of temperature, humidity, and illuminance, continuous value arrays of vital signs, etc.) in real time to a central processing unit via a dedicated data collection module. The central processing unit first performs noise removal (e.g., Butterworth filter, moving average), outlier removal, and normalization (e.g., Z-score normalization) in the data preprocessing unit, and inputs the data to the AI inference module. The AI inference module can use convolutional neural networks (CNN) or recurrent neural networks (RNN) for operation detection, LSTM or GRU for fall detection, and multilayer perceptron (MLP) or decision tree-based ensemble learning (e.g., random forest) for health status analysis. Examples of input to AI include sequences of three-dimensional vectors from acceleration sensors sampled at one-second intervals (e.g., t=0s: [0.12,-0.03, 0.98], t=1s: [0.15,-0.01, 0.97]), continuous value arrays of body temperature, heart rate, and blood pressure every five minutes (e.g., body temperature 36.5° C., heart rate 72 bpm, blood pressure 120/80 mmHg), and binary sequences of motion sensor ON/OFF for each room (e.g., living room: 1, bedroom: 0, hallway: 1). The output of AI is obtained as structured data such as abnormality detection scores (continuous values from 0.0 to 1.0), abnormality type labels (e.g., fall, wandering, vital abnormality), and emergency level determination (e.g., emergency, caution, normal). For example, output examples include “abnormality score 0.92, type: fall, emergency level: emergency” or “abnormality score 0.45, type: vital abnormality, emergency level: caution.” These outputs are compared with preset reference values (e.g., abnormality score of 0.8 or higher) in the threshold determination unit, and if the threshold is exceeded, the data is transferred to the notification unit or instruction unit. The notification unit refers to device information (smartphone, tablet, PC, etc.) and geographic location information (GPS coordinates, address) of family members or caregivers, automatically selects the optimal notification method (push notification, SMS, email, voice call, etc.), and sends the information. The instruction unit automatically instructs actions such as automatic ambulance requests, immediate contact with nearby family members, and contact with care service providers according to the emergency level. Vital sign data from the medical monitoring system is analyzed by AI using time-series abnormality detection algorithms (e.g., autoregressive models, LSTM-based prediction models) to detect sudden changes and deviations from normal patterns with high accuracy.

Furthermore, the system stores past abnormality detection history and response history of family members or caregivers in a database and utilizes them for continuous online learning of AI models and personalized optimization of notification and instruction content. This enables not only automation of human tasks but also integrated improvement of computer technology such as high-dimensional sensor data analysis, cooperation of multiple modules, real-time abnormality detection, automatic notification, and emergency instructions. Technical effects include improved accuracy of abnormality detection (significant reduction of false positives and missed detections), faster response speed (real-time notification and instructions), efficient data management (automatic recording and history analysis), and optimized communication load (notification only when necessary, distributed processing). Specific application fields include monitoring of elderly persons living alone, home medical support, safety management in care facilities, remote health monitoring, support for persons with disabilities, and monitoring of children and pets, among various use cases.

The safety support system according to the embodiment comprises an operation detection unit, an analysis unit, a notification unit, a fall detection unit, an instruction unit, and a health monitoring unit. The operation detection unit detects the operation of an elderly person. For example, the operation detection unit is installed in various locations in a room and monitors the movement of the elderly person in real time. The operation detection unit can detect, for example, walking, standing up, and sitting down. The operation detection unit detects the movement of the elderly person using, for example, motion sensors. The motion sensors are installed in various locations in a room and monitor the movement of the elderly person in real time. For example, the system detects the movement of the elderly person as they move around the room and checks for any abnormalities. The analysis unit analyzes information detected by the operation detection unit. The analysis unit analyzes operation data using AI, for example, and detects abnormalities. The analysis unit can analyze operation data using machine learning algorithms and detect abnormalities. The notification unit provides notifications based on information analyzed by the analysis unit. The notification unit notifies family members or caregivers based on information analyzed by AI, for example. The notification unit notifies family members or caregivers when an abnormality is detected, for example. The fall detection unit detects a fall of an elderly person. The fall detection unit is worn on the body of the elderly person and immediately detects when a fall occurs, for example. The fall detection unit detects a fall using an acceleration sensor, for example. The acceleration sensor is worn on the body of the elderly person and immediately detects when a fall occurs. The instruction unit instructs emergency response based on information analyzed by the analysis unit. The instruction unit instructs emergency response based on information analyzed by AI, for example. The instruction unit instructs actions such as calling an ambulance or contacting family members when a fall is detected, for example. The health monitoring unit monitors the health of the elderly person. The health monitoring unit measures vital signs such as body temperature, heart rate, and blood pressure of the elderly person in real time, for example. The health monitoring unit monitors the health status of the elderly person using a medical monitoring system, for example. The medical monitoring system measures vital signs such as body temperature, heart rate, and blood pressure of the elderly person in real time and sends the information to AI. Thus, the safety support system according to the embodiment can support the safety and health of the elderly person by detecting, analyzing, notifying, and responding to the operation, fall, and health status of the elderly person. Specifically, the safety support system installs multiple types of motion sensors (e.g., infrared sensors, ultrasonic sensors, image sensors) on the ceiling or walls of a room as the operation detection unit, and acquires the position coordinates and operation status (e.g., stationary, walking, pre-fall, etc.) of the elderly person as three-dimensional vector data at one-second intervals. The operation detection unit sends these sensor data as time-series tensors (e.g., a 30×3 matrix for 30 seconds of operation history) to the central processing unit. The analysis unit performs preprocessing such as noise removal (e.g., moving average filter, outlier removal), normalization (Z-score normalization), and then uses convolutional neural networks (CNN) or recurrent neural networks (RNN) for feature extraction and abnormality determination. Examples of input to AI include continuous vector sequences such as t=0s: [0.1, 0.2, 0.0], t=1s: [0.15, 0.18, 0.02], and the output includes structured data such as abnormality scores (0.0-1.0), abnormality types (e.g., fall, wandering, normal), and emergency levels (emergency, caution, normal). The fall detection unit attaches acceleration sensors or gyro sensors to the waist or wrist of the elderly person, samples three-axis acceleration data (e.g., X: 0.98, Y:-0.12, Z: 0.05) at 100 Hz, and analyzes fall-specific patterns (e.g., sudden acceleration changes, posture changes) using time-series AI models such as LSTM or GRU. Examples of AI output from the fall detection unit include “fall score 0.95, emergency level: emergency” or “fall score 0.45, emergency level: caution.” The health monitoring unit obtains vital signs such as body temperature, heart rate, and blood pressure from the medical monitoring system every five minutes, inputs them as continuous value arrays (e.g., body temperature 36.7° C., heart rate 74 bpm, blood pressure 122/78 mmHg) to AI, and uses multilayer perceptron (MLP) or decision tree ensemble (e.g., random forest) for health status analysis, outputting abnormality scores and types (e.g., fever, tachycardia, hypotension). The notification unit receives abnormality determination results from the analysis unit, fall detection unit, and health monitoring unit, compares them with preset reference values (e.g., abnormality score of 0.8 or higher) in the threshold determination unit, and, when the threshold is exceeded, refers to device information (smartphone, tablet, PC, etc.) and geographic location information (GPS coordinates, address) of family members or caregivers to automatically select the optimal notification method (push notification, SMS, email, voice call, etc.) and send the information. The instruction unit automatically instructs actions such as automatic ambulance requests, immediate contact with nearby family members, and contact with care service providers according to the emergency level determination. This series of processes realizes integrated improvement of computer technology such as AI analysis of high-dimensional sensor data, cooperation of multiple modules, real-time abnormality detection, notification, and instructions, which is different from conventional human monitoring and simple threshold determination methods. Technical effects include improved accuracy of abnormality detection (significant reduction of false positives and missed detections), faster response speed (real-time notification and instructions), efficient data management (automatic recording and history analysis), and optimized communication load (notification only when necessary, distributed processing). Specific application fields include monitoring of elderly persons living alone, home medical support, safety management in care facilities, remote health monitoring, support for persons with disabilities, and monitoring of children and pets, among various use cases.

The operation detection unit can be installed in various locations in a room and monitor the movement of the elderly person in real time. For example, the operation detection unit is installed in various locations in a room and monitors the movement of the elderly person in real time. Real time means, for example, updates on a second or minute basis. The operation detection unit detects the movement of the elderly person using motion sensors, for example. The motion sensors are installed in various locations in a room and monitor the movement of the elderly person in real time. For example, the system detects the movement of the elderly person as they move around the room and checks for any abnormalities. By monitoring the movement of the elderly person in real time, abnormalities can be detected early. Specifically, the operation detection unit installs multiple types of motion sensors such as infrared sensors, ultrasonic sensors, and image sensors on the ceiling or walls, and acquires the position coordinates and operation status (e.g., stationary, walking, pre-fall, wandering, etc.) of the elderly person as three-dimensional vector data at one-second intervals or higher frequency. The operation detection unit sends these sensor data as time-series tensors (e.g., a 30×3 matrix for 30 seconds of operation history) to the central processing unit. The operation detection unit performs noise removal (e.g., moving average filter, Butterworth filter), outlier removal, and normalization (e.g., Z-score normalization) in the data preprocessing unit, and inputs the data to the AI inference module. The AI inference module uses convolutional neural networks (CNN) or recurrent neural networks (RNN) for feature extraction and abnormality determination. Examples of input to AI include continuous vector sequences such as t=0s: [0.1, 0.2, 0.0], t=1s: [0.15, 0.18, 0.02], and the output includes structured data such as abnormality scores (0.0-1.0), abnormality types (e.g., fall, wandering, normal), and emergency levels (emergency, caution, normal). For example, output examples include “abnormality score 0.92, type: fall, emergency level: emergency” or “abnormality score 0.45, type: wandering, emergency level: caution.” These outputs are compared with preset reference values (e.g., abnormality score of 0.8 or higher) in the threshold determination unit, and if the threshold is exceeded, the data is transferred to the notification unit or instruction unit. Thus, the operation detection unit realizes integrated improvement of computer technology such as AI analysis of high-dimensional sensor data, cooperation of multiple modules, real-time abnormality detection, notification, and instructions, which is different from conventional human monitoring and simple threshold determination methods. Technical effects include improved accuracy of abnormality detection (significant reduction of false positives and missed detections), faster response speed (real-time notification and instructions), efficient data management (automatic recording and history analysis), and optimized communication load (notification only when necessary, distributed processing). Specific application fields include monitoring of elderly persons living alone, home medical support, safety management in care facilities, remote health monitoring, support for persons with disabilities, and monitoring of children and pets, among various use cases.

The fall detection unit can be worn on the body of the elderly person and immediately detect when a fall occurs. For example, the fall detection unit is worn on the body of the elderly person and immediately detects when a fall occurs. “Immediately” means, for example, detection within a few seconds or a few minutes. The fall detection unit detects a fall using an acceleration sensor, for example. The acceleration sensor is worn on the body of the elderly person and immediately detects when a fall occurs. By immediately detecting a fall, prompt response becomes possible. Specifically, the fall detection unit uses acceleration sensors or gyro sensors that can be attached to the waist or wrist of the elderly person, and continuously acquires three-axis acceleration data (e.g., X: 0.98, Y:-0.12, Z: 0.05) at a high sampling rate such as 100 Hz. The fall detection unit sends these sensor data as time-series tensors (e.g., a 200×3 matrix for two seconds of history) to the central processing unit. The fall detection unit performs noise removal (e.g., low-pass filter), outlier removal, and normalization in the data preprocessing unit, and inputs the data to time-series AI models such as LSTM or GRU. Examples of input to AI include continuous vector sequences such as t=0 ms: [0.98,-0.12, 0.05], t=10 ms: [1.02,-0.10, 0.03], and the output includes structured data such as fall scores (0.0-1.0) and emergency levels (emergency, caution, normal). For example, output examples include “fall score 0.95, emergency level: emergency” or “fall score 0.45, emergency level: caution.” These outputs are compared with preset reference values (e.g., fall score of 0.8 or higher) in the threshold determination unit, and if the threshold is exceeded, the data is transferred to the notification unit or instruction unit. The fall detection unit analyzes fall-specific patterns (e.g., sudden acceleration changes, posture changes) in high-dimensional space using AI models, extracting complex features that differ from simple rule-based human judgment. Thus, compared to conventional human tasks and simple threshold determination methods, technical effects such as improved accuracy of fall detection (significant reduction of false positives and missed detections), faster response speed (real-time notification and instructions), and efficient data management (automatic recording and history analysis) are obtained. Specific application fields include monitoring of elderly persons living alone, safety management in care facilities, support for persons with disabilities, and rehabilitation support.

The health monitoring unit can measure the body temperature, heart rate, and blood pressure of the elderly person in real time. For example, the health monitoring unit measures the body temperature, heart rate, and blood pressure of the elderly person in real time. “Real time” means, for example, updates on a second or minute basis. The health monitoring unit monitors the health status of the elderly person using a medical monitoring system, for example. The medical monitoring system measures vital signs such as body temperature, heart rate, and blood pressure of the elderly person in real time and sends the information to AI. By measuring vital signs in real time, the health status of the elderly person can always be monitored. Specifically, the health monitoring unit attaches medical sensors such as temperature sensors, heart rate sensors, and blood pressure monitors to the body of the elderly person, and continuously acquires vital sign data (e.g., body temperature 36.7° C., heart rate 74 bpm, blood pressure 122/78 mmHg) at set intervals such as every five minutes or every minute. The health monitoring unit sends these data as continuous value arrays or time-series tensors to the central processing unit. The health monitoring unit performs noise removal, outlier removal, and normalization in the data preprocessing unit, and inputs the data to AI models using multilayer perceptron (MLP) or decision tree ensemble (e.g., random forest). Examples of input to AI include continuous value arrays of body temperature, heart rate, and blood pressure every five minutes (e.g., body temperature 36.5° C., heart rate 72 bpm, blood pressure 120/80 mmHg), and one day's worth of vital sign history (288×3 matrix). The output of AI is obtained as structured data such as abnormality scores (0.0-1.0), abnormality types (e.g., fever, tachycardia, hypotension), and emergency levels (emergency, caution, normal). For example, output examples include “abnormality score 0.88, type: fever, emergency level: caution” or “abnormality score 0.95, type: tachycardia, emergency level: emergency.” These outputs are compared with preset reference values in the threshold determination unit, and if the threshold is exceeded, the data is transferred to the notification unit or instruction unit. The health monitoring unit uses time-series abnormality detection algorithms by AI (e.g., autoregressive models, LSTM-based prediction models) to detect sudden changes and deviations from normal patterns with high accuracy. Thus, compared to conventional periodic health checks by humans and simple threshold determination methods, technical effects such as improved accuracy of abnormality detection, faster response speed, and efficient data management are obtained. Specific application fields include health monitoring of elderly persons living alone, home medical support, chronic disease management, health management in care facilities, and remote medical care.

The analysis unit analyzes information from the operation detection unit, fall detection unit, and health monitoring unit, and can notify family members or caregivers when an abnormality is detected. For example, the analysis unit analyzes information from the operation detection unit, fall detection unit, and health monitoring unit, and notifies family members or caregivers when an abnormality is detected. “Abnormality” means, for example, deviation from normal operation patterns or exceeding specific thresholds. The analysis unit analyzes operation data using AI, for example, and detects abnormalities. The analysis unit can analyze operation data using machine learning algorithms and detect abnormalities. By notifying family members or caregivers when an abnormality is detected, prompt response becomes possible. Specifically, the analysis unit integrates time-series operation data from the operation detection unit, acceleration and gyro data from the fall detection unit, and vital sign data from the health monitoring unit, performs noise removal, outlier removal, and normalization in the data preprocessing unit, and inputs the data to the AI inference module. The AI inference module combines different AI architectures such as CNN or RNN for operation analysis, LSTM or GRU for fall detection, and MLP or decision tree ensemble for health status analysis. Examples of input to AI include 30 seconds of operation history (30×3 matrix), two seconds of acceleration history (200×3 matrix), and one day's worth of vital sign history (288×3 matrix). The output of AI is obtained as structured data such as abnormality detection scores (0.0-1.0), abnormality types (e.g., fall, wandering, fever, tachycardia), and emergency level determination (emergency, caution, normal). For example, “abnormality score 0.92, type: fall, emergency level: emergency” or “abnormality score 0.45, type: vital abnormality, emergency level: caution.” These outputs are compared with preset reference values in the threshold determination unit, and if the threshold is exceeded, the data is transferred to the notification unit or instruction unit. The analysis unit stores past abnormality detection history and response history of family members or caregivers in a database and utilizes them for continuous online learning of AI models and personalized optimization of notification and instruction content. This enables not only automation of human tasks but also integrated improvement of computer technology such as high-dimensional sensor data analysis, cooperation of multiple modules, real-time abnormality detection, automatic notification, and emergency instructions. Technical effects include improved accuracy of abnormality detection, faster response speed, efficient data management, and optimized communication load. Specific application fields include monitoring of elderly persons living alone, home medical support, safety management in care facilities, remote health monitoring, and support for persons with disabilities.

The notification unit can notify family members or caregivers based on information analyzed by AI. For example, the notification unit notifies family members or caregivers based on information analyzed by AI. “AI” refers to technologies such as machine learning and deep learning. The notification unit notifies family members or caregivers when an abnormality is detected, for example. By notifying based on information analyzed by AI, accurate information transmission becomes possible. Specifically, the notification unit receives abnormality determination results (e.g., abnormality score, abnormality type, emergency level) from the analysis unit, fall detection unit, and health monitoring unit, compares them with preset reference values (e.g., abnormality score of 0.8 or higher) in the threshold determination unit, and, when the threshold is exceeded, refers to device information (smartphone, tablet, PC, etc.) and geographic location information (GPS coordinates, address) of family members or caregivers to automatically select the optimal notification method (push notification, SMS, email, voice call, etc.) and send the information. The notification unit sends structured data such as abnormality type, occurrence time, emergency level, and recommended response method as notification content. For example, “fall detection: emergency level: emergency, occurrence time: 12:34, recommended response: ambulance request” or “vital abnormality: emergency level: caution, occurrence time: 15:20, recommended response: observation.” The notification unit can also analyze past notification history and response history of family members or caregivers to optimize notification methods, content, and timing for personalization. Thus, compared to conventional simple alert notifications or human phone calls, the accuracy, speed, and personalization of information transmission are greatly improved. Technical effects include reduction of false reports and missed detections, faster response speed, optimized communication load, and improved usability. Specific application fields include monitoring of elderly persons living alone, safety management in care facilities, support for persons with disabilities, and remote health monitoring.

The instruction unit can instruct emergency response based on information analyzed by AI. For example, the instruction unit instructs emergency response based on information analyzed by AI. “Emergency response” means, for example, calling an ambulance or contacting family members. The instruction unit instructs actions such as calling an ambulance or contacting family members when a fall is detected, for example. By instructing emergency response based on information analyzed by AI, prompt response becomes possible. Specifically, the instruction unit receives abnormality determination results (e.g., abnormality score, abnormality type, emergency level) from the analysis unit, fall detection unit, and health monitoring unit, and automatically instructs actions such as automatic ambulance requests, immediate contact with nearby family members, and contact with care service providers according to the emergency level determination. The instruction unit outputs structured data such as abnormality type, occurrence time, recommended response method, and contact information as instruction content, and sends it to the target device or system. For example, “fall detection: ambulance request, family contact, occurrence time: 12:34” or “vital abnormality: observation, family notification, occurrence time: 15:20.” The instruction unit can also analyze past response history and response history of family members or caregivers to optimize response methods, instruction content, and timing for personalization. Thus, compared to conventional human judgment or simple alert notifications, the accuracy, speed, and automation of emergency response are greatly improved. Technical effects include faster response speed, reduction of incorrect responses, efficient data management, and improved usability. Specific application fields include monitoring of elderly persons living alone, safety management in care facilities, support for persons with disabilities, and remote health monitoring.

The notification unit can be equipped with a function for AI to periodically report the status of the elderly person to family members or caregivers. For example, the notification unit is equipped with a function for AI to periodically report the status of the elderly person to family members or caregivers. “Periodically” means, for example, time intervals such as daily or weekly. The notification unit periodically reports the status of the elderly person by AI, for example. By periodically reporting the status of the elderly person, family members or caregivers can feel at ease. Specifically, the notification unit automatically aggregates the elderly person's daily life and health data (e.g., one day's operation history 30×3 matrix, time-series array of vital signs, presence or absence of fall events, emotion estimation results, etc.) collected from the analysis unit, health monitoring unit, operation detection unit, and fall detection unit according to a predetermined schedule (e.g., every day at 8:00 a.m., every Monday at 9:00 a.m.), and inputs it to the AI summary generation module. The AI summary generation module uses, for example, multilayer perceptron (MLP) or time-series feature extraction models (LSTM, Transformer, etc.) to integrate abnormality detection history, health status trends, emotion change tendencies, and response history over the past 24 hours or one week as multidimensional vectors, and generates natural language summary sentences or structured reports (e.g., “No abnormalities detected in the past 24 hours, vital signs are stable, emotion: relaxed tendency”). Examples of input to AI include one day's operation data (1440×3 matrix), vital sign history (288×3 matrix), abnormal event list (e.g., 0 falls, 1 vital abnormality), and emotion estimation scores (e.g., joy 0.2, sadness 0.1, anxiety 0.1, relaxation 0.6). The output of AI is structured data such as summary sentences (e.g., “No abnormalities were detected today. Health status is stable.”), abnormal occurrence time list, and recommended response items. These outputs are automatically sent by the notification unit's scheduler to family members or caregivers' device information (smartphone, tablet, PC, etc.) and preferred notification methods (email, push notification, LINE, etc.). The notification unit can also analyze past notification history and response history of family members or caregivers to optimize notification content and timing for personalization. Technical effects include greatly improved accuracy, efficiency, and peace of mind in information transmission compared to conventional simple periodic reports or manual phone calls, due to AI-based automatic summarization, personalization, and real-time processing of high-dimensional data. Specific application fields include monitoring of elderly persons living alone, home medical support, family contact in care facilities, support for persons with disabilities, and remote health monitoring.

The notification unit can be equipped with a function that enables direct communication between family members or caregivers and the elderly person in an emergency. For example, the notification unit is equipped with a function that enables direct communication between family members or caregivers and the elderly person in an emergency. “Emergency” means, for example, a fall or detection of abnormal vital signs. The notification unit enables direct communication between family members or caregivers and the elderly person in an emergency, for example. By enabling direct communication in an emergency, prompt response becomes possible. Specifically, when the notification unit receives abnormality determination results (e.g., fall score 0.95, emergency level: emergency, abnormality type: vital abnormality, etc.) from the analysis unit, fall detection unit, or health monitoring unit, it immediately links the devices of family members or caregivers (smartphone, tablet, PC, etc.) with the communication terminal at the elderly person's home (e.g., stationary tablet, smart speaker, etc.) and automatically establishes a two-way communication session such as voice call, video call, or text chat. AI can automatically generate explanations of the emergency situation and recommended response content (e.g., “A fall has been detected. Please check the consciousness of the elderly person.”) and provide guidance by voice synthesis at the start of the call. Examples of input to AI include abnormality determination data (abnormality score 0.92, type: fall, emergency level: emergency), current location of the elderly person (e.g., living room), emotion estimation score (e.g., anxiety 0.7), and past response history. The output of AI is structured data such as call initiation triggers, recommended response messages, and call log records. The notification unit records the status of communication session establishment and call content, and saves it in the subsequent response history database. Technical effects include faster, more reliable, and better-recorded information transmission and communication in emergencies compared to conventional simple alert notifications or human phone calls, through the integration of AI abnormality determination and automatic communication control. Specific application fields include monitoring of elderly persons living alone, emergency contact in care facilities, support for persons with disabilities, and emergency response in remote medical settings.

The operation detection unit can estimate the emotion of the elderly person and adjust the sensitivity of operation detection based on the estimated emotion. For example, the operation detection unit estimates the emotion of the elderly person and adjusts the sensitivity of operation detection based on the estimated emotion. “Emotion” means, for example, joy, sadness, anger, etc. If the elderly person is feeling anxious, the operation detection unit increases the sensitivity to detect abnormal operations early. If the elderly person is relaxed, the operation detection unit lowers the sensitivity to reduce false detections. If the elderly person is excited, the operation detection unit appropriately adjusts the sensitivity for accurate operation detection. By adjusting the sensitivity of operation detection according to the emotion of the elderly person, false detections are reduced and accurate detection becomes possible. Specifically, the operation detection unit integrates data obtained from image sensors, audio sensors, and vital sign sensors, and inputs it to the AI emotion estimation module (e.g., multimodal neural network, Transformer-based emotion classifier, etc.). Examples of input to AI include facial expression feature vectors from face images (e.g., 68-point landmark coordinates), spectral features of audio waveforms (e.g., MFCC vectors), heart rate variability data (e.g., five-minute R-R interval array), and operation history over the past 24 hours (1440×3 matrix). The output of AI is structured data such as emotion labels (e.g., joy, sadness, anxiety, relaxation), emotion scores (0.0-1.0), and confidence indicators. The operation detection unit dynamically adjusts the threshold parameters of the operation detection AI (e.g., CNN, RNN) and the sensitivity coefficients of the abnormality detection algorithm based on these emotion estimation results. For example, if the anxiety score is 0.7 or higher, the abnormality detection threshold is lowered from 0.7 to 0.5; if the relaxation score is high, the threshold is raised to 0.8. This enables optimization of false detection reduction and early detection according to the emotional state. Technical effects include improved accuracy, reduced false reports, and improved usability through flexible detection control according to individual states, compared to conventional uniform threshold methods. Specific application fields include monitoring of elderly persons living alone, behavioral monitoring of patients with mental disorders, safety management in care facilities, and support for persons with disabilities.

The operation detection unit can analyze past operation patterns of the elderly person and improve the accuracy of abnormal operation detection. For example, the operation detection unit analyzes past operation patterns of the elderly person and improves the accuracy of abnormal operation detection. “Abnormal operation” means, for example, deviation from normal operation patterns or exceeding specific thresholds. The operation detection unit collects past operation data of the elderly person and learns patterns of abnormal operations. The operation detection unit detects operations that differ from the normal operation patterns of the elderly person and identifies them as abnormal operations. The operation detection unit monitors changes in operation patterns of the elderly person in real time and detects abnormal operations early. By analyzing past operation patterns, the accuracy of abnormal operation detection is improved. Specifically, the operation detection unit stores long-term accumulated operation history data of the elderly person (e.g., one day's worth 1440×3 matrix, one week's worth 10080×3 matrix, etc.) in a database and inputs it to the AI abnormality detection model (e.g., autoencoder, LSTM-based prediction model, clustering algorithm, etc.). Examples of input to AI include operation history tensors for the past 30 days, operation status labels at each time (e.g., walking, stationary, pre-fall, etc.), and abnormal event occurrence time lists. AI learns the distribution of normal patterns and time-series changes, and outputs deviation scores (e.g., 0.0-1.0) and abnormality type labels by comparing newly acquired operation data in real time. For example, output examples include “deviation score 0.85, type: wandering, emergency level: caution” or “deviation score 0.95, type: fall, emergency level: emergency.” These outputs are compared with reference values in the threshold determination unit, and when an abnormality is detected, they are transferred to the notification unit or instruction unit. The operation detection unit uses the online learning function of the AI model to automatically update model parameters by reflecting new daily data, adapting to changes in individual operation characteristics. Technical effects include individual optimization, adaptation to temporal changes, reduction of false detections, and improved detection accuracy compared to conventional fixed rule methods. Specific application fields include monitoring of elderly persons living alone, rehabilitation support, behavioral monitoring of patients with chronic diseases, and safety management in care facilities.

The operation detection unit can optimize detection accuracy based on environmental conditions in the room during operation detection. For example, the operation detection unit optimizes detection accuracy during operation detection by considering environmental conditions in the room (temperature, humidity, lighting, etc.). “Environmental conditions” mean, for example, temperature, humidity, lighting, etc. If the room temperature is high, the operation detection unit adjusts the sensitivity to prevent false detections. If the room humidity is high, the operation detection unit adjusts the sensitivity for accurate detection. If the room lighting is dim, the operation detection unit increases the sensitivity to detect abnormal operations early. By considering environmental conditions in the room, the accuracy of operation detection is improved. Specifically, the operation detection unit obtains time-series data (e.g., temperature, humidity, illuminance values every minute) from environmental sensors (temperature sensor, humidity sensor, illuminance sensor, etc.) and inputs them as additional information to the operation detection AI module. Examples of input to AI include environmental vectors such as t=0 min: [temperature 25.0° C., humidity 60%, illuminance 300 1×], t=1 min: [temperature 25.2° C., humidity 61%, illuminance 280 1×], and operation data at the same time (e.g., labels such as walking, stationary). AI dynamically adjusts abnormality detection thresholds and feature extraction parameters for each environmental condition, for example, suppressing features that are prone to false detection under high temperature and high humidity. The output is structured data such as abnormality score, abnormality type, and emergency level, and optimization for each environmental condition reduces false detections and missed detections. These outputs are compared with reference values in the threshold determination unit and transferred to the notification unit or instruction unit as necessary. Technical effects include improved detection accuracy, reduced false reports, and improved usability under environmental fluctuations compared to conventional environment-unaware detection methods. Specific application fields include monitoring of elderly persons living alone, safety management in care facilities, remote health monitoring, and support for persons with disabilities.

The operation detection unit can estimate the emotion of the elderly person and determine the priority of operation detection based on the estimated emotion. For example, the operation detection unit estimates the emotion of the elderly person and determines the priority of operation detection based on the estimated emotion. “Emotion” means, for example, joy, sadness, anger, etc. If the elderly person is feeling anxious, the operation detection unit increases the priority of operation detection to detect abnormal operations early. If the elderly person is relaxed, the operation detection unit lowers the priority to reduce false detections. If the elderly person is excited, the operation detection unit appropriately adjusts the priority for accurate operation detection. By determining the priority of operation detection according to the emotion of the elderly person, important abnormalities can be detected early. Specifically, the operation detection unit integrates multimodal data obtained from image sensors, audio sensors, and vital sign sensors, and inputs it to the AI emotion estimation module (e.g., multimodal neural network, Transformer-based emotion classifier, etc.). Examples of input to the AI emotion estimation module include facial expression feature vectors (68-point landmark coordinates), MFCC features of audio waveforms, heart rate variability data (five-minute R-R interval array), and operation history over the past 24 hours (1440×3 matrix). AI outputs emotion labels (joy, sadness, anxiety, relaxation, etc.), emotion scores (0.0-1.0), and confidence indicators. For example, output examples include “emotion label: anxiety, score: 0.75” or “emotion label: relaxation, score: 0.85.” The operation detection unit dynamically controls priority parameters and detection scheduling in the abnormality detection algorithm of the operation detection AI (CNN, RNN, etc.) based on emotion estimation results. For example, if the anxiety score is 0.7 or higher, the priority of the abnormality detection process is set to the highest and the detection frequency is increased to one-second intervals. If the relaxation score is high, the priority is lowered and the detection frequency is adjusted to five-second intervals. In the case of excitement, feature extraction parameters are optimized to prevent false detections and the priority of the detection process is set to medium. Examples of AI output include “priority: high, detection interval: 1 second” or “priority: low, detection interval: 5 seconds.” These priority controls are reflected as parameters in the scheduler and AI inference module of the operation detection unit, optimizing resource allocation for the operation detection process and timing of data transfer to the notification and instruction units in real time. Technical effects include early detection of important abnormalities, reduced false reports, and optimized system resources through flexible priority control according to individual emotional states, compared to conventional uniform detection methods. Specific application fields include monitoring of elderly persons living alone, behavioral monitoring of patients with mental disorders, safety management in care facilities, and support for persons with disabilities.

The operation detection unit can adjust the detection range based on the arrangement of furniture in the room during operation detection. For example, the operation detection unit adjusts the detection range during operation detection by considering the arrangement of furniture in the room. “Arrangement of furniture” means, for example, the position and size of furniture. The operation detection unit optimizes the detection range by considering the arrangement of furniture in the room. If the arrangement of furniture changes, the operation detection unit resets the detection range. The operation detection unit adjusts the sensitivity of operation detection according to the arrangement of furniture. By considering the arrangement of furniture in the room, the detection range of operation detection is optimized. Specifically, the operation detection unit obtains furniture arrangement information in the room by spatial scanning with image sensors or by collecting furniture layout data (e.g., coordinates, size, height of each piece of furniture as structured data) through user input. The operation detection unit stores these furniture arrangement data as a three-dimensional spatial map (e.g., room size 6m×4m×2.5m, furniture A: coordinates [1.2, 0.8, 0.0], size [1.0, 0.5, 0.8], etc.) in an internal database. The AI inference module combines the furniture arrangement map and motion sensor arrangement information to automatically calculate the detection range (field of view, blind spots, shielded areas, etc.) for each sensor. For example, if furniture movement or addition is detected, AI identifies new blind spot areas and resets the detection range. Examples of input to AI include furniture arrangement vectors (coordinates and size of each piece of furniture), sensor arrangement vectors (installation position and orientation of each sensor), and past operation detection history (e.g., list of times when detection was missed). The output of AI is structured data such as effective detection range maps for each sensor, blind spot area lists, and detection sensitivity parameters (e.g., sensor 1: sensitivity 0.9, sensor 2: sensitivity 0.7). For example, “sensor 1: detection range [0,2m], blind spot: behind furniture A” or “sensor 2: sensitivity 0.8, detection range [2, 4m].” These outputs are reflected in the sensor control module of the operation detection unit, and the detection range and sensitivity are optimized in real time. If there is a change in furniture arrangement, reconfiguration is performed via the user interface or relearning by automatic scanning. Technical effects include flexible adaptation to changes in furniture arrangement, reduction of blind spots and false detections, improved detection accuracy, and improved usability compared to conventional fixed detection range methods. Specific application fields include monitoring of elderly persons living alone, safety management in care facilities, support for persons with disabilities, and automatic optimization of smart home environments.

The operation detection unit can filter the movement of pets during operation detection to prevent false detection. For example, the operation detection unit filters the movement of pets during operation detection to prevent false detection. “Movement of pets” means, for example, movement patterns and size. The operation detection unit detects the movement of pets and excludes it from the target of operation detection. The operation detection unit distinguishes between the movement of pets and the movement of the elderly person to prevent false detection. The operation detection unit filters the movement of pets to improve the accuracy of operation detection. By filtering the movement of pets, false detection is prevented and the accuracy of operation detection is improved. Specifically, the operation detection unit inputs motion data obtained from image sensors, infrared sensors, ultrasonic sensors, etc. to the AI motion identification module to distinguish between the movement of pets and the movement of the elderly person. Examples of input to AI include object detection feature vectors from image sensors (e.g., bounding box coordinates, object size, movement speed), response patterns from infrared sensors, and past motion history (e.g., pet movement path labels, body height and length information). AI uses convolutional neural networks (CNN) and object detection algorithms (YOLO, SSD, etc.) to classify the type of motion target (elderly person, pet, other) and distinguish between the movement of pets and the movement of the elderly person. The output of AI is structured data such as motion type labels (e.g., elderly person, dog, cat), confidence scores (0.0-1.0), and exclusion flags. For example, “motion type: dog, confidence: 0.92, exclusion: True” or “motion type: elderly person, confidence: 0.98, exclusion: False.” The operation detection unit excludes motion data determined to be pets from the input to the abnormality detection AI to prevent false detection. Furthermore, AI continuously learns the movement patterns of pets (e.g., small movements, low body height, specific movement speed range, etc.) to improve identification accuracy. Thus, compared to conventional simple motion detection methods, technical effects such as significant reduction of false detection due to pets, improved abnormality detection accuracy, and improved usability are achieved. Specific application fields include monitoring of elderly persons living alone, safety management in pet-friendly housing, and animal-assisted care environments in care facilities.

The analysis unit can estimate the emotion of the elderly person and adjust the analysis algorithm based on the estimated emotion. For example, the analysis unit estimates the emotion of the elderly person and adjusts the analysis algorithm based on the estimated emotion. “Emotion” means, for example, joy, sadness, anger, etc. If the elderly person is feeling anxious, the analysis unit increases the sensitivity of the analysis algorithm to detect abnormalities early. If the elderly person is relaxed, the analysis unit lowers the sensitivity to reduce false detections. If the elderly person is excited, the analysis unit appropriately adjusts the sensitivity for accurate analysis. By adjusting the analysis algorithm according to the emotion of the elderly person, analysis accuracy is improved. Specifically, the analysis unit inputs multimodal data obtained from the operation detection unit, health monitoring unit, etc. to the AI emotion estimation module and obtains emotion estimation results (e.g., emotion label, emotion score). Examples of input to AI include facial image feature vectors, audio features, heart rate variability data, and past operation history. The output of AI is structured data such as emotion labels (e.g., anxiety, relaxation, excitement), emotion scores (0.0-1.0), and confidence indicators. The analysis unit dynamically adjusts the sensitivity parameters and thresholds of the abnormality detection AI (CNN, RNN, LSTM, etc.) based on emotion estimation results. For example, if the anxiety score is 0.7 or higher, the abnormality detection threshold is lowered from 0.7 to 0.5; if the relaxation score is high, the threshold is raised to 0.8. In the case of excitement, feature extraction parameters are optimized and filtering is strengthened to prevent false detections. Examples of AI output include “abnormality score 0.92, type: fall, emergency level: emergency” or “abnormality score 0.45, type: vital abnormality, emergency level: caution.” These outputs are compared with reference values in the threshold determination unit and transferred to the notification unit or instruction unit as necessary. Technical effects include improved accuracy, reduced false reports, and improved usability through flexible analysis control according to individual states, compared to conventional uniform threshold methods. Specific application fields include monitoring of elderly persons living alone, behavioral monitoring of patients with mental disorders, safety management in care facilities, and support for persons with disabilities.

The analysis unit can refer to past abnormal detection data during analysis to improve analysis accuracy. For example, the analysis unit refers to past abnormal detection data during analysis to improve analysis accuracy. “Abnormal detection data” means, for example, past abnormal cases or databases. The analysis unit collects past abnormal detection data and optimizes the analysis algorithm. The analysis unit refers to past abnormal detection data and learns patterns of abnormalities. The analysis unit improves the accuracy of the analysis algorithm based on past abnormal detection data. By referring to past abnormal detection data, analysis accuracy is improved. Specifically, the analysis unit stores past abnormal detection history data (e.g., abnormal occurrence time, abnormal type, detection score, response result, etc.) collected from the operation detection unit, fall detection unit, health monitoring unit, etc. in a database and inputs it to the AI abnormality detection model (e.g., autoencoder, LSTM-based prediction model, clustering algorithm, etc.). Examples of input to AI include abnormal detection history tensors for the past 30 days, abnormal event occurrence time lists, and response result labels (e.g., emergency response, observation, etc.). AI learns the distribution of abnormal patterns and time-series changes, and outputs deviation scores and abnormality type labels by comparing newly acquired data in real time. For example, output examples include “deviation score 0.85, type: wandering, emergency level: caution” or “deviation score 0.95, type: fall, emergency level: emergency.” These outputs are compared with reference values in the threshold determination unit, and when an abnormality is detected, they are transferred to the notification unit or instruction unit. The analysis unit uses the online learning function of the AI model to automatically update model parameters by reflecting new daily data, adapting to changes in individual abnormality trends. Technical effects include individual optimization, adaptation to temporal changes, reduction of false detections, and improved detection accuracy compared to conventional fixed rule methods. Specific application fields include monitoring of elderly persons living alone, rehabilitation support, behavioral monitoring of patients with chronic diseases, and safety management in care facilities.

The analysis unit can adjust the level of detail of analysis according to the frequency of abnormal detection during analysis. For example, the analysis unit adjusts the level of detail of analysis according to the frequency of abnormal detection during analysis. “Frequency of abnormal detection” means, for example, the number of detections or time intervals. If the frequency of abnormal detection is high, the analysis unit increases the level of detail of analysis to identify the cause of the abnormality. If the frequency of abnormal detection is low, the analysis unit lowers the level of detail to save resources. The analysis unit dynamically adjusts the level of detail of analysis according to the frequency of abnormal detection. By adjusting the level of detail of analysis according to the frequency of abnormal detection, resource optimization can be achieved. Specifically, the analysis unit aggregates the frequency of abnormal detection events (e.g., number of abnormal detections per hour, interval between abnormal occurrences in the past 24 hours, etc.) received from the operation detection unit, fall detection unit, health monitoring unit, etc. in real time and inputs it to the analysis resolution control module. The analysis resolution control module automatically selects a detailed analysis mode (e.g., high-dimensional feature extraction, abnormality cause estimation, time-series pattern analysis, etc.) when the frequency of abnormal detection is high, and combines multiple algorithms (e.g., abnormality clustering+autoencoder) or deeper neural networks (e.g., deep CNN, LSTM, Transformer, etc.) in the AI inference module for analysis. Examples of input to AI include abnormal occurrence time lists (e.g., t1=10:05, t2=10:12, t3=10:18), abnormal type history (e.g., two falls, one vital abnormality), and time-series tensors of recent operation, vital, and environmental data (e.g., 60 minutes of operation history 3600×3 matrix, vital signs 288×3 matrix). The output of AI is structured data such as abnormality cause estimation labels (e.g., fall cause: unstable walking, vital abnormality cause: fever tendency), abnormality pattern classification (e.g., periodic, sudden, etc.), and detailed abnormality score distribution (e.g., abnormality level 0.0-1.0 for each time). On the other hand, when the frequency of abnormal detection is low, a simple analysis mode (e.g., extraction of only main features, simple threshold determination, lightweight MLP, etc.) is selected to save computational resources and power consumption. These detail controls are reflected as parameters in the scheduler and AI inference module of the analysis unit, optimizing load distribution and real-time performance of the entire system. Technical effects include flexible resource allocation, optimized analysis accuracy, improved computational efficiency, and enhanced system stability according to the abnormality occurrence situation, compared to conventional uniform detail analysis methods. Specific application fields include monitoring of elderly persons living alone, safety management in care facilities, remote health monitoring, support for persons with disabilities, and home medical support.

The analysis unit can estimate the emotion of the elderly person and adjust the display method of the analysis results based on the estimated emotion. For example, the analysis unit estimates the emotion of the elderly person and adjusts the display method of the analysis results according to the estimated emotion. The term ‘emotion’ refers to, for example, joy, sadness, anger, and the like. For instance, when the elderly person is feeling anxious, the analysis unit displays the analysis results in a simple manner to provide reassurance. When the elderly person is relaxed, the analysis unit displays detailed analysis results to deepen understanding. When the elderly person is excited, the analysis unit displays the analysis results in a visually easy-to-understand manner. By adjusting the display method of the analysis results according to the emotion of the elderly person, it becomes possible to provide a display that is easy to understand. Specifically, the analysis unit inputs multimodal data obtained from image sensors, audio sensors, vital sign sensors, and the like into an AI emotion estimation module (e.g., multimodal neural network, Transformer-based emotion classifier, etc.) to estimate emotion labels (e.g., anxiety, relaxation, excitement, etc.) and emotion scores (0.0-1.0). Examples of AI input include facial expression feature vectors (68-point landmark coordinates), MFCC features of audio waveforms, heart rate variability data (5-minute R-R interval arrays), and activity history for the past 24 hours (1440×3 matrix). The AI output is structured data such as emotion labels (e.g., anxiety, relaxation, excitement), emotion scores, and confidence indicators. Based on the emotion estimation results, the analysis unit automatically selects the display mode of the analysis result display module (e.g., simple display, detailed display, visual emphasis display, etc.). For example, if the anxiety score is 0.7 or higher, the abnormality detection result is displayed with a concise message such as ‘No abnormality’ or ‘Safe’ and a large icon to provide reassurance. If the relaxation score is high, time-series graphs of abnormality detection scores, detailed lists of abnormality types, and past response histories are displayed in detail. In the case of excitement, visual emphasis such as color coding and animation is used to present alerts and situation explanations in an easy-to-understand manner. These display controls are also reflected in the layout of the user interface module and the output content to the notification unit. The technical effect is that, compared to conventional uniform display methods, flexible information presentation according to individual emotional states improves understanding, reassurance, prevents misunderstandings, and enhances usability. Specific application fields include monitoring of elderly persons living alone, information provision in nursing care facilities, support for persons with disabilities, and remote health monitoring.

The analysis unit can apply different analysis algorithms according to the type of abnormality detection during analysis. For example, the analysis unit applies different analysis algorithms according to the type of abnormality detection during analysis. The types of abnormality detection refer to, for example, motion abnormalities, vital sign abnormalities, and the like. For instance, in the case of fall detection, the analysis unit applies an analysis algorithm specialized for falls. In the case of health abnormality detection, the analysis unit applies an analysis algorithm specialized for health data. In the case of motion abnormality detection, the analysis unit applies an analysis algorithm specialized for motion data. By applying the optimal analysis algorithm according to the type of abnormality detection, analysis accuracy is improved. Specifically, the analysis unit determines the type of abnormality detection event (e.g., fall, vital abnormality, motion abnormality, etc.) and selects a dedicated AI analysis module for each type. For example, for fall detection, time-series deep learning models such as LSTM or GRU are used, with three-axis acceleration data (e.g., 100 Hz sampling, 2 seconds, 200×3 matrix) as input to output fall scores and urgency. For health abnormality detection, multilayer perceptrons (MLP) or decision tree ensembles (e.g., random forest) are used, with continuous value arrays of vital signs (e.g., body temperature, heart rate, blood pressure, 288×3 matrix) as input to output abnormality scores and abnormality types. For motion abnormality detection, convolutional neural networks (CNN) or recurrent neural networks (RNN) are used, with motion history tensors (e.g., 30 seconds, 30×3 matrix) as input to output abnormality degree and abnormality type. Examples of AI output include ‘fall score 0.95, urgency: emergency’, ‘abnormality score 0.88, type: fever, urgency: caution’, ‘deviation score 0.85, type: wandering, urgency: caution’, and so on. These outputs are compared with reference values in the threshold determination unit and transferred to the notification unit or instruction unit as necessary. The analysis unit optimizes feature extraction methods and determination criteria for each abnormality type and links multiple AI models to greatly improve analysis accuracy and response speed. The technical effect is that, compared to conventional single-algorithm methods, optimization for each abnormality type reduces false detections, improves detection accuracy, and enhances system flexibility. Specific application fields include monitoring of elderly persons living alone, safety management in nursing care facilities, remote health monitoring, support for persons with disabilities, and home medical support.

The analysis unit can determine the priority of analysis based on the time zone of abnormality detection during analysis. For example, the analysis unit determines the priority of analysis based on the time zone of abnormality detection during analysis. The time zone of abnormality detection refers to, for example, daytime, nighttime, and the like. For instance, when an abnormality is detected at night, the analysis unit increases the priority of analysis and responds quickly. When an abnormality is detected during the day, the analysis unit lowers the priority of analysis to save resources. The analysis unit dynamically adjusts the priority of analysis according to the time zone of abnormality detection. By determining the priority of analysis based on the time zone of abnormality detection, rapid response becomes possible. Specifically, the analysis unit records the occurrence time of abnormality detection events (e.g., 24-hour time, day of the week, etc.) as a timestamp and inputs it to the priority control module. The priority control module sets the analysis process priority to the highest level and immediately allocates AI inference module resources and transfers data to the notification and instruction units when an abnormality is detected during specific time zones such as nighttime (e.g., 22:00-6:00) or holidays. During daytime or normal time zones, the priority is lowered, batch processing or simplified analysis mode is selected, and system load and power consumption are optimized. Examples of AI input include abnormal occurrence time lists (e.g., t1=23:15, t2=02:30), abnormality types (e.g., fall, vital abnormality), and past response histories. The AI output is structured data such as priority labels (high, medium, low), recommended response timing (immediate, normal, delayed, etc.), and analysis scheduling information. These priority controls are reflected in the scheduler of the analysis unit and the operation timing of the notification and instruction units, enabling immediate response to nighttime emergency abnormalities and resource saving for minor daytime abnormalities. The technical effect is that, compared to conventional uniform priority methods, flexible priority control according to time zone enables rapid response, resource optimization, and improved system stability. Specific application fields include monitoring of elderly persons living alone, nighttime safety management in nursing care facilities, remote health monitoring, and support for persons with disabilities.

The notification unit can estimate the emotion of the elderly person and adjust the content of the notification based on the estimated emotion. For example, the notification unit estimates the emotion of the elderly person and adjusts the content of the notification according to the estimated emotion. The term ‘emotion’ refers to, for example, joy, sadness, anger, and the like. For instance, when the elderly person is feeling anxious, the notification unit provides a notification with content that gives reassurance. When the elderly person is relaxed, the notification unit provides a notification containing detailed information. When the elderly person is excited, the notification unit provides a notification with concise and easy-to-understand content. By adjusting the content of the notification according to the emotion of the elderly person, appropriate information transmission becomes possible. Specifically, the notification unit inputs multimodal data obtained from image sensors, audio sensors, vital sign sensors, and the like into an AI emotion estimation module to estimate emotion labels (e.g., anxiety, relaxation, excitement, etc.) and emotion scores (0.0-1.0). Examples of AI input include facial image feature vectors, audio feature quantities, heart rate variability data, and past activity history. The AI output is structured data such as emotion labels, emotion scores, and confidence indicators. Based on the emotion estimation results, the notification unit automatically selects templates and expression methods of the notification content generation module. For example, if the anxiety score is 0.7 or higher, a reassuring message such as ‘The current situation is safe. Please rest assured.’ is generated. If the relaxation score is high, a detailed notification including abnormality detection scores, health status details, and recommended actions is created. In the case of excitement, concise and easy-to-understand expressions (e.g., ‘A fall has been detected. Please be careful.’) are used. The notification unit also analyzes notification history and response history of family members or caregivers to optimize personalization of notification content. The technical effect is that, compared to conventional uniform notification methods, flexible information transmission according to individual emotional states improves reassurance, understanding, prevents misunderstandings, and enhances usability. Specific application fields include monitoring of elderly persons living alone, information provision in nursing care facilities, support for persons with disabilities, and remote health monitoring.

The notification unit can refer to the past response history of family members or caregivers during notification to optimize the notification method. For example, the notification unit refers to the past response history of family members or caregivers during notification to optimize the notification method. The past response history refers to, for example, responses to notifications, response history, and the like. The notification unit analyzes the past response history of family members or caregivers and selects the optimal notification method. For instance, the notification unit prioritizes notification methods to which family members or caregivers responded quickly. The notification unit adjusts the content and timing of notifications based on the response history of family members or caregivers. By referring to past response history, the optimal notification method can be selected. Specifically, the notification unit constructs a notification history database for each family member or caregiver, recording the response content to each notification event (e.g., notification receipt time, app launch, response button press, phone call, on-site visit, etc.), time required to complete response, notification means (push notification, SMS, email, voice call, etc.), notification content (abnormality type, urgency, recommended response, etc.) in chronological order. The notification unit inputs these history data into an AI notification optimization module (e.g., gradient boosting decision tree, MLP, time-series clustering, etc.) to learn response tendencies and optimal notification means, timing, and content for each family member or caregiver. Examples of AI input include notification event history for the past 30 days (e.g., notification type, means, response time, response result, 288×5 matrix), device usage tendencies for each family member (e.g., smartphone usage rate 0.9, PC usage rate 0.1), and past emergency response history (e.g., 10 emergency notifications, average response time 3 minutes). The AI output is structured data such as recommended notification means (e.g., push notification, SMS, voice call), recommended notification timing (e.g., immediate, 5 minutes later, refrain at night, etc.), and notification content templates (e.g., detailed notification, simple notification). For example, ‘Family A: push notification, immediate, detailed content’, ‘Caregiver B: SMS, 5 minutes later, simple content’, and so on. Based on the AI output, the notification unit controls the notification scheduler and content generation module to automatically select and send the optimal notification method, timing, and content for each family member or caregiver. Furthermore, response data after notification is continuously collected, and the optimization accuracy is improved by online learning of the AI model. As a result, compared to conventional uniform notification methods or manual selection by humans, notification optimization according to individual response tendencies enables faster response, reduction of erroneous or missed notifications, improved usability, and optimized communication load. Specific application fields include monitoring of elderly persons living alone, family communication in nursing care facilities, support for persons with disabilities, and remote health monitoring.

The notification unit can adjust the urgency of notifications according to the type of abnormality during notification. For example, the notification unit adjusts the urgency of notifications according to the type of abnormality during notification. The types of abnormality refer to, for example, motion abnormalities, vital sign abnormalities, and the like. For instance, when a fall is detected, the notification unit provides a highly urgent notification. When a health abnormality is detected, the notification unit adjusts the content of the notification according to the urgency. When a motion abnormality is detected, the notification unit adjusts the timing of the notification according to the urgency. By adjusting the urgency of notifications according to the type of abnormality, rapid response becomes possible. Specifically, the notification unit inputs abnormality determination data (e.g., abnormality type label, abnormality score, urgency determination value, etc.) received from the analysis unit, fall detection unit, and health monitoring unit into the notification determination module, and applies urgency determination rules and AI classification models (e.g., decision tree, MLP, rule-based threshold determination, etc.) for each abnormality type. Examples of AI input include structured data such as ‘abnormality type: fall, abnormality score: 0.95’, ‘abnormality type: vital abnormality, abnormality score: 0.88’, ‘abnormality type: motion abnormality, abnormality score: 0.75’, and so on. The AI output includes urgency labels (e.g., emergency, caution, normal), recommended notification timing (e.g., immediate, 5 minutes later, at scheduled report time, etc.), and notification content templates (e.g., detailed notification, simple notification). For example, ‘fall: emergency, immediate notification’, ‘vital abnormality: caution, detailed notification’, ‘motion abnormality: normal, scheduled report’, and so on. Based on the AI output, the notification unit controls the notification scheduler and content generation module to automatically select and send the optimal urgency, notification timing, and content for each abnormality type. Furthermore, the notification unit analyzes past response histories and family/caregiver reaction histories for each abnormality type to continuously optimize the parameters of urgency determination rules and AI models. As a result, compared to conventional uniform urgency notification methods, optimization for each abnormality type enables faster response, reduction of false reports and missed notifications, improved usability, and efficient use of system resources. Specific application fields include monitoring of elderly persons living alone, safety management in nursing care facilities, support for persons with disabilities, and remote health monitoring.

The notification unit can estimate the emotion of the elderly person and adjust the timing of notifications based on the estimated emotion. For example, the notification unit estimates the emotion of the elderly person and adjusts the timing of notifications according to the estimated emotion. The term ‘emotion’ refers to, for example, joy, sadness, anger, and the like. For instance, when the elderly person is feeling anxious, the notification unit provides notifications promptly. When the elderly person is relaxed, the notification unit delays the timing of notifications. When the elderly person is excited, the notification unit provides notifications at an appropriate timing. By adjusting the timing of notifications according to the emotion of the elderly person, notifications can be provided at the appropriate timing. Specifically, the notification unit inputs multimodal data obtained from image sensors, audio sensors, vital sign sensors, and the like into an AI emotion estimation module (e.g., multimodal neural network, Transformer-based emotion classifier, etc.) to estimate emotion labels (e.g., anxiety, relaxation, excitement, etc.) and emotion scores (0.0-1.0). Examples of AI input include facial expression feature vectors (68-point landmark coordinates), MFCC features of audio waveforms, heart rate variability data (5-minute R-R interval arrays), and activity history for the past 24 hours (1440×3 matrix). The AI output is structured data such as emotion labels, emotion scores, and confidence indicators. Based on the emotion estimation results, the notification unit dynamically controls the timing parameters of the notification scheduler. For example, if the anxiety score is 0.7 or higher, immediate notification is executed upon abnormality detection; if the relaxation score is high, the notification timing is delayed by 5 to 10 minutes. In the case of excitement, notifications are provided at a timing when the situation has stabilized to prevent misunderstanding or confusion. Examples of AI output include ‘notification timing: immediate’, ‘notification timing: 5 minutes later’, ‘notification timing: after situation stabilizes’, and so on. These timing controls are reflected in the scheduler and content generation module of the notification unit, enabling real-time optimization of the notification process. The technical effect is that, compared to conventional uniform notification timing methods, flexible notification timing control according to individual emotional states improves reassurance, reduces false reports, and enhances usability. Specific application fields include monitoring of elderly persons living alone, behavioral monitoring of patients with mental disorders, information provision in nursing care facilities, and support for persons with disabilities.

The notification unit can select the optimal notification method by considering the geographic location information of family members or caregivers during notification. For example, the notification unit selects the optimal notification method by considering the geographic location information of family members or caregivers during notification. Geographic location information refers to, for example, GPS information, addresses, and the like. For instance, when family members or caregivers are nearby, the notification unit provides notifications that encourage direct visits. When family members or caregivers are far away, the notification unit provides notifications by phone or message. The notification unit selects the optimal notification method based on the geographic location information of family members or caregivers. By selecting the optimal notification method based on geographic location information, rapid response becomes possible. Specifically, the notification unit creates a database of the latest geographic location information (e.g., GPS coordinates, addresses, movement history, etc.) for each family member or caregiver and obtains real-time location information via a location information acquisition module when a notification event occurs. The notification unit inputs the current location of the notification target, the location of the abnormality occurrence (e.g., room information at the elderly person's home), and past response history (e.g., number of on-site visits, number of remote responses, etc.) into an AI notification means selection module (e.g., decision tree, MLP, rule-based determination, etc.). Examples of AI input include ‘Family A: GPS coordinates [35.6, 139.7], elderly person's home: GPS coordinates [35.6, 139.8], distance: 1.2 km’, ‘Caregiver B: address: Tokyo, distance: 10 km’, and so on. The AI output is structured data such as recommended notification means (e.g., direct visit encouragement, phone, SMS, email), notification content templates (e.g., on-site visit request, remote response request, etc.), and notification priority (high, medium, low). For example, ‘Family A: direct visit recommended, immediate notification’, ‘Caregiver B: phone notification, detailed content’, and so on. Based on the AI output, the notification unit controls the notification scheduler and content generation module to automatically select and send the optimal notification method, content, and timing according to geographic location information. Furthermore, response history after notification is continuously collected, and the optimization accuracy is improved by online learning of the AI model. As a result, compared to conventional uniform notification means methods, notification optimization utilizing geographic location information enables faster response, reduction of erroneous or missed notifications, improved usability, and optimized communication load. Specific application fields include monitoring of elderly persons living alone, family communication in nursing care facilities, support for persons with disabilities, and remote health monitoring.

The notification unit can adjust the format of notifications by considering the device information of family members or caregivers during notification. For example, the notification unit adjusts the format of notifications by considering the device information of family members or caregivers during notification. Device information refers to, for example, smartphones, tablets, and the like. For instance, when family members or caregivers are using smartphones, the notification unit provides push notifications. When family members or caregivers are using personal computers, the notification unit provides notifications by email. The notification unit selects the optimal notification format based on the device information of family members or caregivers. By adjusting the format of notifications based on device information, appropriate information transmission becomes possible. Specifically, the notification unit constructs a device usage history database for each family member or caregiver, recording the device type (e.g., smartphone, tablet, PC, feature phone, etc.), OS type, app usage status, notification reception settings, etc., for each notification event. The notification unit inputs the latest device information of the notification target, past notification reception history, response history, etc., into an AI notification format selection module (e.g., decision tree, MLP, rule-based determination, etc.). Examples of AI input include ‘Family A: smartphone usage rate 0.95, PC usage rate 0.05’, ‘Caregiver B: tablet usage rate 0.8, notification app installed’, and so on. The AI output is structured data such as recommended notification format (e.g., push notification, SMS, email, voice call), notification content templates (e.g., detailed notification, simple notification), and notification priority (high, medium, low). For example, ‘Family A: push notification, detailed content’, ‘Caregiver B: email notification, simple content’, and so on. Based on the AI output, the notification unit controls the notification scheduler and content generation module to automatically select and send the optimal notification format, content, and timing according to device information. Furthermore, reception and response history after notification is continuously collected, and the optimization accuracy is improved by online learning of the AI model. As a result, compared to conventional uniform notification format methods, notification optimization utilizing device information enables more accurate and rapid information transmission, improved usability, and optimized communication load. Specific application fields include monitoring of elderly persons living alone, family communication in nursing care facilities, support for persons with disabilities, and remote health monitoring.

The fall detection unit can estimate the emotion of the elderly person and adjust the sensitivity of fall detection based on the estimated emotion. For example, the fall detection unit estimates the emotion of the elderly person and adjusts the sensitivity of fall detection according to the estimated emotion. The term ‘emotion’ refers to, for example, joy, sadness, anger, and the like. For instance, when the elderly person is feeling anxious, the fall detection unit increases the sensitivity of fall detection to detect abnormalities early. When the elderly person is relaxed, the fall detection unit lowers the sensitivity to reduce false detections. When the elderly person is excited, the fall detection unit appropriately adjusts the sensitivity for accurate detection. By adjusting the sensitivity of fall detection according to the emotion of the elderly person, false detections are reduced and accurate detection becomes possible. Specifically, the fall detection unit inputs data obtained from image sensors, audio sensors, vital sign sensors, and the like into an AI emotion estimation module to obtain emotion estimation results (e.g., emotion label, emotion score). Examples of AI input include facial image feature vectors, audio feature quantities, heart rate variability data, and past activity history. The AI output is structured data such as emotion labels (e.g., anxiety, relaxation, excitement), emotion scores (0.0-1.0), and confidence indicators. Based on the emotion estimation results, the fall detection unit dynamically adjusts the sensitivity parameters and thresholds of the fall detection AI (LSTM, GRU, etc.). For example, if the anxiety score is 0.7 or higher, the fall detection threshold is lowered from 0.7 to 0.5; if the relaxation score is high, the threshold is raised to 0.8. In the case of excitement, feature extraction parameters are optimized and filtering is strengthened to prevent false detections. Examples of AI output include ‘fall score 0.95, urgency: emergency’ and ‘fall score 0.45, urgency: caution’. These outputs are compared with reference values in the threshold determination unit and transferred to the notification unit or instruction unit as necessary. The technical effect is that, compared to conventional uniform threshold methods, flexible detection control according to individual states improves accuracy, reduces false reports, and enhances usability. Specific application fields include monitoring of elderly persons living alone, behavioral monitoring of patients with mental disorders, safety management in nursing care facilities, and support for persons with disabilities.

The fall detection unit can analyze the past fall history of the elderly person to improve the accuracy of fall detection. For example, the fall detection unit analyzes the past fall history of the elderly person to improve the accuracy of fall detection. Fall history refers to, for example, past fall cases, databases, and the like. The fall detection unit collects past fall data of the elderly person and learns fall patterns. The fall detection unit identifies situations with a high risk of falls based on the past fall history of the elderly person. The fall detection unit analyzes the fall history of the elderly person and optimizes the fall detection algorithm. By analyzing past fall history, the accuracy of fall detection is improved. Specifically, the fall detection unit accumulates detailed data of past fall events (e.g., occurrence time, location, acceleration sensor values, vital sign changes, activity history immediately before the fall, etc.) as time-series tensors or structured event lists in a database. The fall detection unit inputs these history data into an AI abnormality detection model (e.g., LSTM-based time-series prediction model, autoencoder, clustering algorithm, etc.) to learn characteristic patterns and risk factors (e.g., decreased walking speed, unstable posture, sudden changes in vital signs, etc.) in high-dimensional space immediately before a fall occurs. Examples of AI input include fall event history tensors for the past 30 days (e.g., 200×3 acceleration matrix for each event, vital sign history, environmental condition vectors), fall occurrence time lists, and activity state labels before and after falls (e.g., walking, stationary, pre-fall signs, etc.). The AI output is structured data such as fall risk scores (0.0-1.0), risk factor labels (e.g., unstable walking, environmental factors, poor physical condition, etc.), and fall pattern classification (e.g., sudden type, gradual type, etc.). For example, ‘fall risk score 0.85, factor: unstable walking’, ‘fall risk score 0.92, factor: poor physical condition’, and so on. Based on these AI outputs, the fall detection unit individually optimizes the threshold parameters and feature extraction methods of the real-time fall detection AI (LSTM, GRU, etc.), and increases sensitivity for early detection when movements or situations similar to past fall patterns are detected. Furthermore, the fall history database is equipped with online learning functions, and the AI model parameters are automatically updated to reflect new fall events and near-miss cases on a daily basis, adapting to changes in individual risk tendencies. As a result, compared to conventional fixed rule methods or simple threshold determination methods, individual optimization, adaptation to temporal changes, reduction of false detections, and improvement of detection accuracy are achieved. Technical effects include improved accuracy of fall detection (significant reduction of false detections and missed detections), faster response speed (real-time notification and instruction), efficient data management (automatic recording and history analysis), and enhanced system flexibility. Specific application fields include monitoring of elderly persons living alone, safety management in nursing care facilities, rehabilitation support, fall risk management for patients with chronic diseases, and support for persons with disabilities.

The fall detection unit can optimize detection accuracy based on the physical condition and clothing of the elderly person during fall detection. For example, the fall detection unit optimizes detection accuracy by considering the physical condition and clothing of the elderly person during fall detection. Physical condition refers to, for example, degree of fatigue, medical history, and the like. Clothing refers to, for example, type of shoes, thickness of clothing, and the like. For instance, when the physical condition of the elderly person is poor, the fall detection unit increases sensitivity. When the clothing of the elderly person makes movement difficult, the fall detection unit adjusts sensitivity. The fall detection unit optimizes the fall detection algorithm according to the physical condition and clothing of the elderly person. By considering the physical condition and clothing of the elderly person, the accuracy of fall detection is improved. Specifically, the fall detection unit collects physical condition data (e.g., subjective fatigue score, vital sign abnormality history, medical history information, medication status, etc.) and clothing data (e.g., shoe type label, clothing thickness/mobility index, wearable sensor information, etc.) obtained from the health monitoring unit or user interface as structured data. The fall detection unit inputs these physical condition and clothing data into an AI fall risk estimation module (e.g., multilayer perceptron MLP, decision tree ensemble, rule-based determination, etc.) to calculate fall risk scores and sensitivity adjustment parameters. Examples of AI input include vital sign history for the past 24 hours (288×3 matrix), fatigue score (0.0-1.0), shoe type (e.g., sneakers, slippers, sandals, etc.), clothing thickness (mm), mobility index (0.0-1.0), and so on. The AI output is structured data such as fall risk scores (0.0-1.0), sensitivity adjustment values (e.g., +0.2,-0.1, etc.), and recommended detection algorithms (e.g., high sensitivity mode, normal mode, etc.). For example, ‘fall risk 0.85, sensitivity +0.2’, ‘fall risk 0.45, sensitivity −0.1’, and so on. Based on the AI output, the fall detection unit dynamically adjusts the thresholds and feature extraction parameters of the fall detection AI (LSTM, GRU, etc.), increasing sensitivity for early detection when physical condition is poor or clothing makes movement difficult, and suppressing false detections when physical condition is good and clothing allows easy movement. Furthermore, physical condition and clothing data are accumulated as daily history in a database, and the AI model adapts to changes in individual tendencies through online learning. As a result, compared to conventional uniform threshold methods or subjective human judgment, flexible detection control considering physical condition and clothing improves accuracy, reduces false reports, and enhances usability. Technical effects include improved accuracy of fall detection, reduction of false detections and missed detections, enhanced system flexibility, and individual optimization. Specific application fields include monitoring of elderly persons living alone, safety management in nursing care facilities, rehabilitation support, fall risk management for patients with chronic diseases, and support for persons with disabilities.

The fall detection unit can estimate the emotion of the elderly person and determine the priority of fall detection based on the estimated emotion. For example, the fall detection unit estimates the emotion of the elderly person and determines the priority of fall detection according to the estimated emotion. The term ‘emotion’ refers to, for example, joy, sadness, anger, and the like. For instance, when the elderly person is feeling anxious, the fall detection unit increases the priority of fall detection. When the elderly person is relaxed, the fall detection unit lowers the priority. When the elderly person is excited, the fall detection unit appropriately adjusts the priority. By determining the priority of fall detection according to the emotion of the elderly person, important abnormalities can be detected early. Specifically, the fall detection unit inputs multimodal data obtained from image sensors, audio sensors, vital sign sensors, and the like (e.g., facial image feature vectors, MFCC features of audio waveforms, heart rate variability data, activity history for the past 24 hours, etc.) into an AI emotion estimation module (e.g., multimodal neural network, Transformer-based emotion classifier, etc.) to estimate emotion labels (e.g., anxiety, relaxation, excitement, etc.) and emotion scores (0.0-1.0). Examples of AI input include facial expression feature vectors (68-point landmark coordinates), audio features, heart rate variability data (5-minute R-R interval arrays), and activity history for the past 24 hours (1440×3 matrix). The AI output is structured data such as emotion labels, emotion scores, and confidence indicators. For example, ‘emotion label: anxiety, score: 0.75’, ‘emotion label: relaxation, score: 0.85’, and so on. Based on the emotion estimation results, the fall detection unit dynamically controls the priority parameters and detection scheduling of the fall detection AI (LSTM, GRU, etc.). For example, if the anxiety score is 0.7 or higher, the priority of the fall detection process is set to the highest level and the detection frequency is increased to 1-second intervals. If the relaxation score is high, the priority is lowered and the detection frequency is adjusted to 5-second intervals. In the case of excitement, feature extraction parameters are optimized to prevent false detections and the priority of the detection process is set to a medium level. Examples of AI output include ‘priority: high, detection interval: 1 second’, ‘priority: low, detection interval: 5 seconds’, and so on. These priority controls are reflected as parameters in the scheduler and AI inference module of the fall detection unit, optimizing resource allocation for the fall detection process and timing of data transfer to the notification and instruction units in real time. The technical effect is that, compared to conventional uniform detection methods, flexible priority control according to individual emotional states enables early detection of important abnormalities, reduction of false reports, and optimization of system resources. Specific application fields include monitoring of elderly persons living alone, behavioral monitoring of patients with mental disorders, safety management in nursing care facilities, and support for persons with disabilities.

The fall detection unit can adjust detection accuracy based on the material and condition of the floor during fall detection. For example, the fall detection unit adjusts detection accuracy by considering the material and condition of the floor during fall detection. The material of the floor refers to, for example, carpet, flooring, and the like. The condition of the floor refers to, for example, being wet, slippery, and the like. For instance, when the floor is slippery, the fall detection unit increases sensitivity. When the floor is hard, the fall detection unit adjusts sensitivity. The fall detection unit optimizes the fall detection algorithm according to the material and condition of the floor. By considering the material and condition of the floor, the accuracy of fall detection is improved. Specifically, the fall detection unit collects floor material data (e.g., labels for carpet, flooring, tatami, etc.) and floor condition data (e.g., wetness sensor values, slipperiness index, temperature/humidity values, etc.) obtained from environmental sensors or user interface as structured data. The fall detection unit inputs these floor information into an AI fall risk estimation module (e.g., decision tree, MLP, rule-based determination, etc.) to calculate fall risk scores and sensitivity adjustment parameters. Examples of AI input include floor material labels (e.g., carpet=0, flooring=1), floor condition vectors (wetness 0.8, slipperiness 0.7), environmental conditions (temperature, humidity), and so on. The AI output is structured data such as fall risk scores (0.0-1.0), sensitivity adjustment values (e.g., +0.2,-0.1, etc.), and recommended detection algorithms (e.g., high sensitivity mode, normal mode, etc.). For example, ‘fall risk 0.9, sensitivity+0.3’, ‘fall risk 0.4, sensitivity −0.1’, and so on. Based on the AI output, the fall detection unit dynamically adjusts the thresholds and feature extraction parameters of the fall detection AI (LSTM, GRU, etc.), increasing sensitivity for early detection on slippery or wet floors, and suppressing false detections on hard or safe floors. Floor material and condition data are accumulated as history in a database, and the AI model adapts to environmental changes through online learning. As a result, compared to conventional environment-unaware detection, flexible detection control considering floor conditions improves accuracy, reduces false reports, and enhances usability. Technical effects include improved accuracy of fall detection, reduction of false detections and missed detections, enhanced system flexibility, and individual optimization. Specific application fields include monitoring of elderly persons living alone, safety management in nursing care facilities, rehabilitation support, support for persons with disabilities, and automatic optimization in smart home environments.

The fall detection unit can instruct different responses according to the impact level of the fall during fall detection. For example, the fall detection unit instructs different responses according to the impact level of the fall during fall detection. The impact level refers to, for example, values from acceleration sensors, strength of impact, and the like. For instance, when the impact level of the fall is high, the fall detection unit instructs emergency response. When the impact level of the fall is low, the fall detection unit instructs mild response. The fall detection unit instructs appropriate responses according to the impact level of the fall. By instructing different responses according to the impact level of the fall, appropriate response becomes possible. Specifically, the fall detection unit inputs three-axis acceleration data obtained from acceleration sensors and gyro sensors during fall events (e.g., 100 Hz sampling, 2 seconds, 200×3 matrix) into an AI impact level estimation module (e.g., convolutional neural network CNN, decision tree, rule-based determination, etc.) to calculate impact level scores (0.0-1.0) and classification labels (e.g., high impact, low impact, etc.). Examples of AI input include peak acceleration values at the time of fall (e.g., 2.5G), impact duration (e.g., 0.3 seconds), changes in vital signs (e.g., sudden increase in heart rate), and activity state immediately before the fall. The AI output is structured data such as impact level scores (0.0-1.0), impact level classification (high, medium, low), and recommended response labels (e.g., emergency response, observation, mild response, etc.). For example, ‘impact level 0.95, high, emergency response’, ‘impact level 0.45, low, mild response’, and so on. Based on the AI output, the fall detection unit automatically generates response instructions according to the impact level (e.g., request for ambulance, contact family, observation, etc.) for the instruction unit or notification unit and sends them to the target devices or systems. Furthermore, past impact levels and response result histories are accumulated in a database, and the optimization accuracy is improved by online learning of the AI model. As a result, compared to conventional uniform response methods or subjective human judgment, flexible response instructions considering impact level enable faster and more accurate response and reduction of erroneous responses. Technical effects include faster response speed, reduction of erroneous responses, improved usability, and enhanced system flexibility. Specific application fields include monitoring of elderly persons living alone, safety management in nursing care facilities, rehabilitation support, support for persons with disabilities, and remote health monitoring.

The instruction unit can estimate the emotion of the elderly person and adjust the content of emergency response instructions based on the estimated emotion. For example, the instruction unit estimates the emotion of the elderly person and adjusts the content of emergency response instructions according to the estimated emotion. The term ‘emotion’ refers to, for example, joy, sadness, anger, and the like. For instance, when the elderly person is feeling anxious, the instruction unit provides instructions that give reassurance. When the elderly person is relaxed, the instruction unit provides detailed instructions. When the elderly person is excited, the instruction unit provides concise and easy-to-understand instructions. By adjusting the content of emergency response instructions according to the emotion of the elderly person, appropriate response becomes possible. Specifically, the instruction unit inputs multimodal data obtained from image sensors, audio sensors, vital sign sensors, and the like (e.g., facial expression feature vectors, MFCC features of audio waveforms, heart rate variability data, activity history for the past 24 hours, etc.) into an AI emotion estimation module (e.g., multimodal neural network, Transformer-based emotion classifier, etc.). Examples of input to the AI emotion estimation module include 68-point landmark coordinates of facial images, MFCC feature vectors of audio, 5-minute R-R interval arrays, and 1440×3 matrices of activity history. The AI output is structured data such as emotion labels (e.g., anxiety, relaxation, excitement), emotion scores (0.0-1.0), and confidence indicators. For example, ‘emotion label: anxiety, score: 0.78’, ‘emotion label: relaxation, score: 0.85’, and so on. Based on the emotion estimation results, the instruction unit automatically selects templates and expression methods of the emergency response instruction generation module. For example, if the anxiety score is 0.7 or higher, a reassuring message such as ‘The current situation is safe. Please rest assured. An ambulance has been requested.’ is generated. If the relaxation score is high, detailed instructions including abnormality detection scores, health status details, and recommended actions (e.g., ‘A fall has been detected. Ambulance requested, family notified. Please remain calm until arrival.’) are created. In the case of excitement, concise and easy-to-understand expressions (e.g., ‘Fall detected. Ambulance requested. Remain calm.’) are used. Examples of AI output include ‘instruction content: reassurance type, detailed type, concise type’, and so on. These instruction contents are sent to the devices of family members or caregivers and the terminal at the elderly person's home via the output module of the instruction unit. Furthermore, instruction content history and response history of the elderly person and family are analyzed, and personalization optimization of instruction content is performed by online learning of the AI model. As a result, compared to conventional uniform instruction methods, flexible instruction content generation according to individual emotional states improves reassurance, understanding, prevents misunderstandings, and enhances usability. Technical effects include improved accuracy, speed, and personalization of emergency response, reduction of erroneous responses, and enhanced system flexibility. Specific application fields include monitoring of elderly persons living alone, emergency response in nursing care facilities, support for persons with disabilities, and remote health monitoring.

The instruction unit can refer to past response history during emergency response to select the optimal response method. For example, the instruction unit refers to past response history during emergency response to select the optimal response method. Past response history refers to, for example, response methods, results, and the like. The instruction unit analyzes past emergency response history and selects the optimal response method. The instruction unit proposes rapid response methods based on past response history. The instruction unit selects appropriate response methods by referring to past response history. By referring to past response history, the optimal response method can be selected. Specifically, the instruction unit accumulates history data of past emergency response events (e.g., ambulance requests during falls, family contact, observation, etc.) (e.g., occurrence time, response method, time required to complete response, result label, response content of family members or caregivers, etc.) as a time-series database. The instruction unit inputs these history data into an AI response optimization module (e.g., gradient boosting decision tree, MLP, time-series clustering, etc.) to learn response patterns, success rates, speed, and response tendencies for each family member or caregiver. Examples of AI input include response history for the past 30 days (e.g., response type, means, response time, result, 288×5 matrix), response success rate for each family member (e.g., ambulance request success rate 0.95, family contact success rate 0.9), and past emergency response history (e.g., 10 emergency responses, average response time 5 minutes). The AI output is structured data such as recommended response methods (e.g., ambulance request, family contact, observation), recommended response means (e.g., phone, SMS, on-site visit), and recommended response timing (e.g., immediate, 5 minutes later), and so on. For example, ‘recommended response: ambulance request, immediate’, ‘recommended response: family contact, phone’, and so on. Based on the AI output, the instruction unit controls the instruction content generation module to automatically select and output the optimal response method, means, and timing based on past history. Furthermore, result data after response is continuously collected, and the optimization accuracy is improved by online learning of the AI model. As a result, compared to conventional uniform response methods or manual selection by humans, response optimization utilizing individual and situational history enables faster response, reduction of erroneous responses and missed responses, improved usability, and efficient use of system resources. Specific application fields include monitoring of elderly persons living alone, emergency response in nursing care facilities, support for persons with disabilities, and remote health monitoring.

The instruction unit can adjust the level of detail of response according to the type of abnormality during emergency response. For example, the instruction unit adjusts the level of detail of response according to the type of abnormality during emergency response. The types of abnormality refer to, for example, motion abnormalities, vital sign abnormalities, and the like. For instance, when a fall is detected, the instruction unit provides detailed response instructions. When a health abnormality is detected, the instruction unit adjusts the level of detail of response according to the type of abnormality. When a motion abnormality is detected, the instruction unit adjusts the level of detail of response according to the type of abnormality. By adjusting the level of detail of response according to the type of abnormality, appropriate response becomes possible. Specifically, the instruction unit inputs abnormality determination data (e.g., abnormality type label, abnormality score, urgency determination value, etc.) received from the analysis unit, fall detection unit, health monitoring unit, and the like into the instruction content generation module, and applies detail level determination rules and AI classification models (e.g., decision tree, MLP, rule-based determination, etc.) for each abnormality type. Examples of AI input include structured data such as ‘abnormality type: fall, abnormality score: 0.95’, ‘abnormality type: vital abnormality, abnormality score: 0.88’, ‘abnormality type: motion abnormality, abnormality score: 0.75’, and so on. The AI output includes response detail level labels (e.g., detailed, normal, simple), recommended instruction content templates (e.g., detailed instructions, simple instructions), and recommended response means (e.g., ambulance request, family contact, observation), and so on. For example, ‘fall: detailed instructions, ambulance request’, ‘vital abnormality: normal instructions, family contact’, ‘motion abnormality: simple instructions, observation’, and so on. Based on the AI output, the instruction unit controls the instruction content generation module to automatically select and output the optimal detail level, instruction content, and response means for each abnormality type. Furthermore, the instruction unit analyzes past response histories and family/caregiver reaction histories for each abnormality type to continuously optimize the parameters of detail level determination rules and AI models. As a result, compared to conventional uniform detail level instruction methods, optimization for each abnormality type enables faster response, reduction of erroneous responses and missed responses, improved usability, and efficient use of system resources. Specific application fields include monitoring of elderly persons living alone, emergency response in nursing care facilities, support for persons with disabilities, and remote health monitoring.

The instruction unit can estimate the emotion of the elderly person and determine the priority of emergency response based on the estimated emotion. For example, the instruction unit estimates the emotion of the elderly person and determines the priority of emergency response according to the estimated emotion. The term ‘emotion’ refers to, for example, joy, sadness, anger, and the like. For instance, when the elderly person is feeling anxious, the instruction unit increases the priority of emergency response. When the elderly person is relaxed, the instruction unit lowers the priority. When the elderly person is excited, the instruction unit appropriately adjusts the priority. By determining the priority of emergency response according to the emotion of the elderly person, important abnormalities can be responded to early. Specifically, the instruction unit inputs multimodal data obtained from image sensors, audio sensors, vital sign sensors, and the like (e.g., facial image feature vectors, MFCC features of audio waveforms, heart rate variability data, activity history for the past 24 hours, etc.) into an AI emotion estimation module (e.g., multimodal neural network, Transformer-based emotion classifier, etc.) to estimate emotion labels (e.g., anxiety, relaxation, excitement, etc.) and emotion scores (0.0-1.0). Examples of AI input include 68-point landmark coordinates of facial images, audio features, 5-minute R-R interval arrays, and 1440×3 matrices of activity history. The AI output is structured data such as emotion labels, emotion scores, and confidence indicators. For example, ‘emotion label: anxiety, score: 0.75’, ‘emotion label: relaxation, score: 0.85’, and so on. Based on the emotion estimation results, the instruction unit dynamically controls the priority parameters and response scheduling of the emergency response priority control module. For example, if the anxiety score is 0.7 or higher, the priority of the emergency response process is set to the highest level and the frequency of issuing response instructions is increased to 1-minute intervals. If the relaxation score is high, the priority is lowered and the frequency of issuing response instructions is adjusted to 5-minute intervals. In the case of excitement, feature extraction parameters are optimized to prevent erroneous responses and the priority of the response process is set to a medium level. Examples of AI output include ‘priority: high, response interval: 1 minute’, ‘priority: low, response interval: 5 minutes’, and so on. These priority controls are reflected as parameters in the scheduler and AI inference module of the instruction unit, optimizing resource allocation for the emergency response process and timing of data transfer to the notification and instruction units in real time. The technical effect is that, compared to conventional uniform response methods, flexible priority control according to individual emotional states enables early response to important abnormalities, reduction of erroneous responses, and optimization of system resources. Specific application fields include monitoring of elderly persons living alone, emergency response for patients with mental disorders, safety management in nursing care facilities, and support for persons with disabilities.

The instruction unit can select the optimal response method by considering the geographic location information of family members or caregivers during emergency response. For example, the instruction unit selects the optimal response method by considering the geographic location information of family members or caregivers during emergency response. Geographic location information refers to, for example, GPS information, addresses, and the like. For instance, when family members or caregivers are nearby, the instruction unit selects a response method that encourages direct visits. When family members or caregivers are far away, the instruction unit instructs response by phone or message. The instruction unit selects the optimal response method based on the geographic location information of family members or caregivers. By selecting the optimal response method based on geographic location information, rapid response becomes possible. Specifically, the instruction unit creates a database of the latest geographic location information (e.g., GPS coordinates, addresses, movement history, etc.) for each family member or caregiver and obtains real-time location information via a location information acquisition module when an emergency response event occurs. The instruction unit inputs the current location of the response target, the location of the abnormality occurrence (e.g., room information at the elderly person's home), and past response history (e.g., number of on-site visits, number of remote responses, etc.) into an AI response means selection module (e.g., decision tree, MLP, rule-based determination, etc.). Examples of AI input include ‘Family A: GPS coordinates [35.6, 139.7], elderly person's home: GPS coordinates [35.6, 139.8], distance: 1.2 km’, ‘Caregiver B: address: Tokyo, distance: 10 km’, and so on. The AI output is structured data such as recommended response means (e.g., direct visit encouragement, phone, SMS, email), response content templates (e.g., on-site visit request, remote response request, etc.), and response priority (high, medium, low). For example, ‘Family A: direct visit recommended, immediate response’, ‘Caregiver B: phone response, detailed content’, and so on. Based on the AI output, the instruction unit controls the response scheduler and content generation module to automatically select and output the optimal response method, content, and timing according to geographic location information. Furthermore, response history after response is continuously collected, and the optimization accuracy is improved by online learning of the AI model. As a result, compared to conventional uniform response means methods, response optimization utilizing geographic location information enables faster response, reduction of erroneous responses and missed responses, improved usability, and efficient use of system resources. Specific application fields include monitoring of elderly persons living alone, emergency response in nursing care facilities, support for persons with disabilities, and remote health monitoring.

The instruction unit can adjust the format of response by considering the device information of family members or caregivers during emergency response. For example, during emergency response, the instruction unit adjusts the response format based on the device information of family members or caregivers. Device information refers to, for example, smartphones, tablets, and the like. If a family member or caregiver is using a smartphone, the instruction unit instructs the response via push notification. If a family member or caregiver is using a personal computer, the instruction unit instructs the response via email. The instruction unit selects the optimal response format based on the device information of family members or caregivers. By adjusting the response format according to the device information of family members or caregivers, appropriate information transmission can be achieved. Specifically, the instruction unit constructs a device usage history database for each family member or caregiver, recording the device type (e.g., smartphone, tablet, PC, feature phone, etc.), OS type, application usage status, notification reception settings, and so on for each response event. The instruction unit inputs the latest device information of the target person, past response history, and response records into an AI response format selection module (e.g., decision tree, MLP, rule-based judgment, etc.). Examples of AI input include “Family member A: smartphone usage rate 0.95, PC usage rate 0.05” and “Caregiver B: tablet usage rate 0.8, notification app installed.” The AI output is structured data such as recommended response format (e.g., push notification, SMS, email, voice call), response content template (e.g., detailed instructions, simple instructions), and response priority (high, medium, low). For example, “Family member A: push notification, detailed content” and “Caregiver B: email notification, simple content.” Based on the AI output, the instruction unit controls the response scheduler and content generation module, automatically selecting and outputting the optimal response format, content, and timing according to the device information. Furthermore, the instruction unit continuously collects post-response reception and reply history, improving optimization accuracy through online learning of the AI model. As a result, compared to conventional uniform response format methods, technical effects such as improved accuracy, speed, and usability of information transmission and system resource efficiency are achieved by optimizing response using device information. Specific application fields include monitoring of elderly persons living alone, emergency response in care facilities, support for persons with disabilities, and remote health monitoring.

The health monitoring unit can estimate the emotion of an elderly person and adjust the sensitivity of health monitoring based on the estimated emotion. For example, the health monitoring unit estimates the emotion of an elderly person and adjusts the sensitivity of health monitoring according to the estimated emotion. Emotion refers to, for example, joy, sadness, anger, and the like. If the elderly person is feeling anxious, the health monitoring unit increases the sensitivity to detect abnormalities early. If the elderly person is relaxed, the health monitoring unit lowers the sensitivity to reduce false detections. If the elderly person is excited, the health monitoring unit appropriately adjusts the sensitivity for accurate monitoring. By adjusting the sensitivity of health monitoring according to the emotion of the elderly person, false detections are reduced and accurate monitoring is enabled. Specifically, the health monitoring unit inputs multimodal data obtained from image sensors, audio sensors, vital sign sensors, etc. (e.g., facial expression feature vectors, MFCC features of audio waveforms, heart rate variability data, activity history for the past 24 hours) into an AI emotion estimation module (e.g., multimodal neural network, Transformer-based emotion classifier). Examples of input to the AI emotion estimation module include 68-point facial landmark coordinates, MFCC feature vectors of audio, 5-minute R-R interval arrays, and 1440×3 matrices of activity history. The AI output is structured data such as emotion labels (e.g., anxiety, relaxation, excitement), emotion scores (0.0-1.0), and confidence indicators. For example, “Emotion label: anxiety, score: 0.78” and “Emotion label: relaxation, score: 0.85.” Based on the emotion estimation results, the health monitoring unit dynamically adjusts the sensitivity parameters and thresholds of the health monitoring AI (e.g., LSTM, GRU, CNN). For example, if the anxiety score is 0.7 or higher, the health monitoring threshold is lowered from 0.7 to 0.5; if the relaxation score is high, the threshold is raised to 0.8. In the case of excitement, feature extraction parameters are optimized and filtering is strengthened to prevent false detections. Examples of AI output include “Abnormality score 0.92, type: vital abnormality, urgency: emergency” and “Abnormality score 0.45, type: mild abnormality, urgency: caution.” These outputs are compared with reference values in the threshold judgment unit and transferred to the notification unit or instruction unit as necessary. The health monitoring unit automatically updates model parameters by reflecting new daily data through online learning of the AI model, adapting to individual emotional changes and health status changes. Technical effects include improved accuracy, reduced false alarms, and enhanced usability through flexible monitoring control according to individual conditions and emotional states, compared to conventional uniform threshold methods. Specific application fields include monitoring of elderly persons living alone, health monitoring of patients with mental disorders, safety management in care facilities, support for persons with disabilities, and home medical support.

The health monitoring unit can analyze past health data of an elderly person and improve the accuracy of abnormality detection. For example, the health monitoring unit analyzes past health data of an elderly person to improve the accuracy of abnormality detection. Health data refers to, for example, past health records, vital sign data, and the like. The health monitoring unit collects past health data of the elderly person and learns patterns of abnormalities. Based on past health data, the health monitoring unit identifies situations with a high risk of abnormality. The health monitoring unit analyzes health data of the elderly person and optimizes the abnormality detection algorithm. By analyzing past health data, the accuracy of abnormality detection is improved. Specifically, the health monitoring unit accumulates previously obtained vital sign data (e.g., body temperature, heart rate, blood pressure, respiratory rate as a 288×4 matrix per day), health records (e.g., medication history, medical history, subjective health scores), and abnormal event history (e.g., abnormal occurrence time, abnormal type, response result) as time-series tensors or structured databases. The health monitoring unit inputs these historical data into an AI abnormality detection model (e.g., LSTM-based time-series prediction model, autoencoder, clustering algorithm) to learn characteristic patterns and risk factors (e.g., periodic increase in heart rate, sudden change in body temperature, abnormal blood pressure fluctuations) in high-dimensional space just before abnormality occurrence. Examples of AI input include a tensor of vital sign history for the past 30 days (288×4×30), a list of abnormal occurrence times, and health status labels (e.g., good, caution, abnormal). The AI output is structured data such as abnormality risk score (0.0-1.0), risk factor labels (e.g., arrhythmia, fever tendency, blood pressure fluctuation), and abnormality pattern classification (e.g., periodic type, sudden type). For example, “Abnormality risk score 0.85, factor: arrhythmia” and “Abnormality risk score 0.92, factor: fever tendency.” Based on these AI outputs, the health monitoring unit individually optimizes threshold parameters and feature extraction methods of real-time health monitoring AI (LSTM, GRU, etc.), increasing sensitivity for early detection when situations similar to past abnormal patterns are detected. Furthermore, the health database is equipped with online learning functionality, automatically updating AI model parameters by reflecting new daily health events and near-miss cases, and adapting to changes in individual risk tendencies. As a result, compared to conventional fixed rule or simple threshold judgment methods, individual optimization, adaptation to temporal changes, reduction of false detections, and improved detection accuracy are achieved. Technical effects include improved accuracy of health monitoring (significant reduction of false detections and missed detections), faster response speed (real-time notification and instruction), efficient data management (automatic recording and history analysis), and enhanced system flexibility. Specific application fields include monitoring of elderly persons living alone, health management in care facilities, rehabilitation support, health risk management for patients with chronic diseases, and support for persons with disabilities.

The health monitoring unit can optimize monitoring accuracy based on seasonal and weather influences during health monitoring. For example, the health monitoring unit optimizes monitoring accuracy by considering seasonal and weather influences during health monitoring. Season refers to, for example, summer, winter, and the like. Weather refers to, for example, sunny, rainy, and the like. The health monitoring unit adjusts the sensitivity of health monitoring at seasonal changes to detect abnormalities early. If the weather is bad, the health monitoring unit increases the sensitivity. The health monitoring unit optimizes the health monitoring algorithm according to seasonal and weather influences. By considering seasonal and weather influences, the accuracy of health monitoring is improved. Specifically, the health monitoring unit collects seasonal information (e.g., month/date, average temperature, sunshine duration) and weather information (e.g., sunny, rain, humidity, atmospheric pressure, outdoor temperature as time-series data) obtained from environmental sensors or external weather APIs as structured data. The health monitoring unit inputs these environmental data as additional information into the health monitoring AI (e.g., decision tree, MLP, rule-based judgment), dynamically adjusting abnormality detection thresholds and feature extraction parameters for each season and weather. Examples of AI input include environmental vectors such as “Season: summer, outdoor temperature: 32° C., humidity: 80%” and “Weather: rain, pressure: 990 hPa, indoor temperature: 28° C.,” along with simultaneous vital sign data (e.g., body temperature, heart rate, blood pressure). The AI output is structured data such as abnormality score, abnormality type, and urgency, and optimization for each season and weather reduces false detections and missed detections. For example, “Abnormality score 0.88, type: heatstroke risk, urgency: caution” and “Abnormality score 0.92, type: hypothermia risk, urgency: emergency.” These outputs are compared with reference values in the threshold judgment unit and transferred to the notification unit or instruction unit as necessary. The health monitoring unit optimizes AI model parameters through online learning according to environmental changes, flexibly responding to seasonal diseases and weather-dependent risks. Technical effects include improved monitoring accuracy, reduced false alarms, and enhanced usability under environmental fluctuations compared to conventional environment-unaware monitoring. Specific application fields include monitoring of elderly persons living alone, health management in care facilities, remote health monitoring, support for persons with disabilities, and home medical support.

The health monitoring unit can estimate the emotion of an elderly person and determine the priority of health monitoring based on the estimated emotion. For example, the health monitoring unit estimates the emotion of an elderly person and determines the priority of health monitoring according to the estimated emotion. Emotion refers to, for example, joy, sadness, anger, and the like. If the elderly person is feeling anxious, the health monitoring unit raises the priority of health monitoring. If the elderly person is relaxed, the health monitoring unit lowers the priority. If the elderly person is excited, the health monitoring unit appropriately adjusts the priority. By determining the priority of health monitoring according to the emotion of the elderly person, important abnormalities can be detected early. Specifically, the health monitoring unit inputs multimodal data obtained from image sensors, audio sensors, vital sign sensors, etc. (e.g., facial image feature vectors, MFCC features of audio waveforms, heart rate variability data, activity history for the past 24 hours) into an AI emotion estimation module (e.g., multimodal neural network, Transformer-based emotion classifier) to estimate emotion labels (e.g., anxiety, relaxation, excitement) and emotion scores (0.0-1.0). Examples of AI input include facial expression feature vectors (68-point landmark coordinates), audio features, heart rate variability data (5-minute R-R interval arrays), and activity history for the past 24 hours (1440×3 matrix). The AI output is structured data such as emotion label, emotion score, and confidence indicator. For example, “Emotion label: anxiety, score: 0.75” and “Emotion label: relaxation, score: 0.85.” Based on the emotion estimation results, the health monitoring unit dynamically controls priority parameters and monitoring scheduling of the health monitoring AI (LSTM, GRU, etc.). For example, if the anxiety score is 0.7 or higher, the priority of the health monitoring process is set to the highest and the monitoring frequency is increased to 1-minute intervals. If the relaxation score is high, the priority is lowered and the monitoring frequency is adjusted to 5-minute intervals. In the case of excitement, feature extraction parameters are optimized to prevent false detections and the priority of the monitoring process is set to medium. Examples of AI output include “Priority: high, monitoring interval: 1 minute” and “Priority: low, monitoring interval: 5 minutes.” These priority controls are reflected as parameters in the scheduler and AI inference module of the health monitoring unit, optimizing resource allocation for the health monitoring process and timing of data transfer to the notification and instruction units in real time. Technical effects include early detection of important abnormalities, reduced false alarms, and optimized system resources through flexible priority control according to individual emotional states, compared to conventional uniform monitoring methods. Specific application fields include monitoring of elderly persons living alone, health monitoring of patients with mental disorders, safety management in care facilities, and support for persons with disabilities.

The health monitoring unit can improve the accuracy of abnormality detection during health monitoring by referring to health data of family members or caregivers. For example, during health monitoring, the health monitoring unit refers to health data of family members or caregivers to improve the accuracy of abnormality detection. Health data of family members or caregivers refers to, for example, past health records, vital sign data, and the like. The health monitoring unit refers to health data of family members or caregivers and learns patterns of abnormalities. Based on health data of family members or caregivers, the health monitoring unit identifies situations with a high risk of abnormality. The health monitoring unit analyzes health data of family members or caregivers and optimizes the abnormality detection algorithm. By referring to health data of family members or caregivers, the accuracy of abnormality detection is improved. Specifically, the health monitoring unit accumulates health records of family members or caregivers (e.g., vital sign history, medical history, genetic risk information, lifestyle data) as time-series tensors or structured databases. The health monitoring unit inputs these data into an AI abnormality detection model (e.g., decision tree, MLP, clustering algorithm) to learn common abnormal patterns and genetic risk factors (e.g., tendency for high blood pressure, risk of heart disease) among family members and caregivers. Examples of AI input include vital sign history for three family members over the past year (365×4×3 matrix), medical history labels (e.g., hypertension, diabetes), and lifestyle scores (e.g., exercise frequency, dietary balance). The AI output is structured data such as abnormality risk score (0.0-1.0), risk factor labels (e.g., genetic hypertension, lifestyle disease risk), and abnormality pattern classification (e.g., family common type, individual specific type). For example, “Abnormality risk 0.85, factor: familial hypertension” and “Abnormality risk 0.92, factor: lifestyle disease.” Based on these AI outputs, the health monitoring unit optimizes threshold parameters and feature extraction methods of real-time health monitoring AI according to the risk tendencies of family members and caregivers, increasing sensitivity for early detection when genetic or lifestyle risk is high. Furthermore, the health database of family members and caregivers is equipped with online learning functionality, automatically updating AI model parameters by reflecting new daily data and adapting to changes in risk tendencies. As a result, compared to conventional individual-only monitoring methods, risk prediction, reduction of false detections, and improved detection accuracy are achieved by utilizing health information of family members and caregivers. Technical effects include improved accuracy of health monitoring, reduced false detections and missed detections, enhanced system flexibility, and health risk management at the family unit level. Specific application fields include monitoring of elderly persons living alone, family unit health management, health risk assessment in care facilities, early detection of hereditary diseases, and support for persons with disabilities.

The health monitoring unit can apply different monitoring algorithms according to the type of abnormality during health monitoring. For example, during health monitoring, the health monitoring unit applies different monitoring algorithms according to the type of abnormality. Types of abnormality refer to, for example, operation abnormality, vital sign abnormality, and the like. If a body temperature abnormality is detected, the health monitoring unit applies a monitoring algorithm specialized for body temperature. If a heart rate abnormality is detected, the health monitoring unit applies a monitoring algorithm specialized for heart rate. If a blood pressure abnormality is detected, the health monitoring unit applies a monitoring algorithm specialized for blood pressure. By applying the optimal monitoring algorithm according to the type of abnormality, monitoring accuracy is improved. Specifically, the health monitoring unit determines the type of abnormality detection event (e.g., body temperature abnormality, heart rate abnormality, blood pressure abnormality, operation abnormality) and selects a dedicated AI monitoring module for each type. For example, for body temperature abnormality detection, a time-series LSTM or decision tree is used, with body temperature data (e.g., 288 points per day at 1-minute intervals) as input, outputting abnormality score and urgency. For heart rate abnormality detection, a CNN or RNN is used, with heart rate variability data (e.g., 5-minute R-R interval arrays, 288×1 matrix) as input, outputting abnormality score and abnormality type. For blood pressure abnormality detection, a multilayer perceptron (MLP) or random forest is used, with blood pressure data (e.g., 288×2 matrix) as input, outputting abnormality score and abnormality type. Examples of AI output include “Body temperature abnormality score 0.95, urgency: emergency,” “Heart rate abnormality score 0.88, type: arrhythmia, urgency: caution,” and “Blood pressure abnormality score 0.85, type: hypertension, urgency: caution.” These outputs are compared with reference values in the threshold judgment unit and transferred to the notification unit or instruction unit as necessary. The health monitoring unit optimizes feature extraction methods and judgment criteria for each type of abnormality and links multiple AI models to greatly improve monitoring accuracy and response speed. Technical effects include reduced false detections, improved detection accuracy, and enhanced system flexibility through optimization for each type of abnormality, compared to conventional single-algorithm methods. Specific application fields include monitoring of elderly persons living alone, health management in care facilities, remote health monitoring, support for persons with disabilities, and home medical support.

The system according to the embodiment is not limited to the examples described above and may be variously modified as follows, for example.

The operation detection unit can monitor the temperature and humidity of a room in real time and automatically adjust the sensitivity of operation detection when abnormal environmental conditions are detected. For example, if the room temperature rises rapidly, the operation detection unit increases the sensitivity to detect abnormal operations early. If the humidity is high, the sensitivity of operation detection is adjusted to prevent false detections. As a result, operation detection according to environmental conditions is enabled, and more accurate monitoring can be achieved.

The fall detection unit can predict the fall risk of an elderly person and adjust the sensitivity of fall detection based on the predicted risk. For example, if the elderly person has a history of falling, the fall detection unit increases the sensitivity to detect another fall early. If the elderly person is in poor physical condition, the fall risk increases, so the sensitivity can be further increased. As a result, fall detection according to fall risk is enabled, and the safety of the elderly person can be more reliably ensured.

The health monitoring unit can collect data on the diet and exercise of an elderly person and evaluate the health status based on these data. For example, the health monitoring unit monitors whether the elderly person is consuming a balanced diet and notifies family members or caregivers if a nutritional deficiency is detected. If a lack of exercise is detected, the health monitoring unit can send a notification to encourage appropriate exercise. As a result, by utilizing data on diet and exercise, the health status of the elderly person can be comprehensively monitored.

The analysis unit can estimate the emotion of an elderly person and adjust the display method of analysis results based on the estimated emotion. For example, if the elderly person is feeling anxious, the analysis results are displayed simply to provide reassurance. If the elderly person is relaxed, detailed analysis results are displayed to deepen understanding. Furthermore, if the elderly person is excited, the analysis results can be displayed in a visually easy-to-understand manner. As a result, analysis results can be displayed according to the emotion of the elderly person, enabling the provision of easily understandable information.

The notification unit can optimize the notification method by referring to the past response history of family members or caregivers. For example, by prioritizing notification methods to which family members or caregivers responded quickly, emergency responses can be performed promptly. The content and timing of notifications can also be adjusted based on past response history. As a result, by utilizing past response history, the optimal notification method can be selected, enabling prompt and appropriate information transmission.

The instruction unit can estimate the emotion of an elderly person and adjust the content of emergency response instructions based on the estimated emotion. For example, if the elderly person is feeling anxious, the instruction unit provides instructions that offer reassurance. If the elderly person is relaxed, the instruction unit provides detailed instructions to deepen understanding. Furthermore, if the elderly person is excited, the instruction unit provides concise and easy-to-understand instructions. As a result, emergency response instructions can be provided according to the emotion of the elderly person, enabling appropriate response.

The health monitoring unit can optimize monitoring accuracy based on seasonal and weather influences. For example, the health monitoring unit adjusts the sensitivity at seasonal changes to detect abnormalities early. If the weather is bad, the sensitivity of health monitoring can be increased. As a result, health monitoring that considers seasonal and weather influences is enabled, and more accurate abnormality detection can be achieved.

The operation detection unit can filter the movement of pets to prevent false detections. For example, the operation detection unit distinguishes between the movement of pets and the movement of the elderly person and excludes pets from the detection target. The operation detection unit can also learn patterns of pet movement to reduce false detections. As a result, by filtering the movement of pets, the accuracy of operation detection is improved and false detections can be prevented.

The notification unit can estimate the emotion of an elderly person and adjust the content of notifications based on the estimated emotion. For example, if the elderly person is feeling anxious, the notification unit sends notifications with content that provides reassurance. If the elderly person is relaxed, the notification unit sends notifications containing detailed information. Furthermore, if the elderly person is excited, the notification unit sends notifications with concise and easy-to-understand content. As a result, notifications can be provided according to the emotion of the elderly person, enabling appropriate information transmission.

The instruction unit can select the optimal response method by considering the geographic location information of family members or caregivers during emergency response. For example, if family members or caregivers are nearby, the instruction unit selects a response method that encourages direct visits. If family members or caregivers are far away, the instruction unit can instruct response via phone or message. As a result, the optimal response method can be selected based on the geographic location information of family members or caregivers, enabling prompt response.

The processing flow of Example of the Embodiment will be briefly described below.

Step 1: The operation detection unit detects the movement of an elderly person. For example, the operation detection unit is installed in various locations in a room and monitors walking, standing up, sitting down, and other movements of the elderly person in real time using motion sensors. Step 2: The analysis unit analyzes information detected by the operation detection unit. For example, the analysis unit analyzes motion data using AI or machine learning algorithms to detect abnormalities. Step 3: The notification unit provides notifications based on information analyzed by the analysis unit. For example, if an abnormality is detected, the notification unit notifies family members or caregivers. Step 4: The fall detection unit detects a fall of the elderly person. For example, the fall detection unit is attached to the body of the elderly person using an acceleration sensor and immediately detects when a fall occurs. Step 5: The instruction unit instructs emergency response based on information analyzed by the analysis unit. For example, if a fall is detected, the instruction unit instructs calling an ambulance or contacting family members. Step 6: The health monitoring unit monitors the health of the elderly person. For example, the health monitoring unit measures vital signs such as body temperature, heart rate, and blood pressure of the elderly person in real time and transmits the information to AI using a medical monitoring system. Specifically, in Step 1, the system installs multiple motion sensors (infrared sensors, ultrasonic sensors, image sensors, etc.) on the ceiling or walls and acquires the position coordinates and movement status (stationary, walking, pre-fall, etc.) of the elderly person as three-dimensional vector data at one-second intervals. In Step 2, these sensor data are sent to a central processing unit as time-series tensors (e.g., a 30×3 matrix for 30 seconds of movement history), where a data preprocessing unit performs noise removal (moving average filter, outlier removal) and normalization (Z-score normalization), followed by feature extraction and abnormality determination using convolutional neural networks (CNN) or recurrent neural networks (RNN). Examples of AI input include continuous vector sequences such as t=0s: [0.1, 0.2, 0.0], t=1s: [0.15, 0.18, 0.02], and the output includes structured data such as abnormality score (0.0-1.0), abnormality type (fall, wandering, normal), and urgency (emergency, caution, normal). In Step 3, the notification unit receives abnormality determination results from the analysis unit, fall detection unit, and health monitoring unit, compares them with preset reference values (abnormality score of 0.8 or higher) in the threshold judgment unit, and, when the threshold is exceeded, refers to device information (smartphone, tablet, PC, etc.) and geographic location information (GPS coordinates, address) of family members or caregivers to automatically select the optimal notification method (push notification, SMS, email, voice call, etc.) and transmit the information. In Step 4, the fall detection unit samples three-axis acceleration data at 100 Hz from acceleration sensors or gyro sensors attached to the waist or wrist of the elderly person and analyzes fall-specific patterns using time-series AI models such as LSTM or GRU. Examples of AI output include “Fall score 0.95, urgency: emergency” and “Fall score 0.45, urgency: caution.” In Step 5, the instruction unit automatically instructs actions such as automatic ambulance requests, immediate contact with nearby family members, and contact with care service providers according to the urgency determination. In Step 6, the health monitoring unit obtains vital signs such as body temperature, heart rate, and blood pressure from the medical monitoring system every five minutes, inputs them as continuous value arrays into AI, and outputs abnormality scores and abnormality types (fever, tachycardia, hypotension). This series of processing, unlike conventional human monitoring or simple threshold judgment methods, realizes integrated improvement of computer technology through AI analysis of high-dimensional sensor data, cooperation of multiple modules, and real-time abnormality detection, notification, and instruction. Technical effects include improved accuracy of abnormality detection (significant reduction of false detections and missed detections), faster response speed (real-time notification and instruction), efficient data management (automatic recording and history analysis), and optimized communication load (notification only when necessary, distributed processing). Specific application fields include monitoring of elderly persons living alone, home medical support, safety management in care facilities, remote health monitoring, support for persons with disabilities, and monitoring of children or pets, among various use cases.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai. com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned operation detection unit, analysis unit, notification unit, fall detection unit, instruction unit, and health monitoring unit is implemented by at least one of, for example, a smart device 14 and a data processing apparatus 12. For example, the operation detection unit detects the movement of an elderly person using a motion sensor of the smart device 14. The analysis unit is implemented, for example, by a specific processing unit 290 of the data processing apparatus 12 and analyzes operation data. The notification unit is implemented, for example, by a control unit 46A of the smart device 14 and notifies family members or caregivers when an abnormality is detected. The fall detection unit detects a fall of the elderly person using, for example, an acceleration sensor of the smart device 14. The instruction unit is implemented, for example, by the specific processing unit 290 of the data processing apparatus 12 and instructs emergency response. The health monitoring unit monitors the health status of the elderly person using, for example, a medical monitoring system of the smart device 14. The correspondence between each unit and the device or control unit is not limited to the examples described above and various modifications are possible.

### Second Embodiment

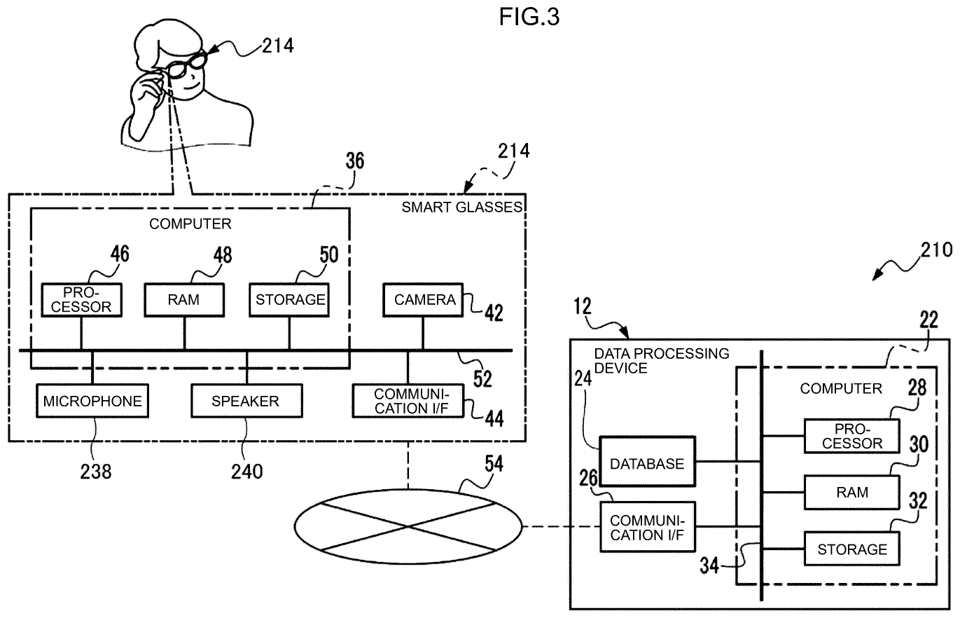

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

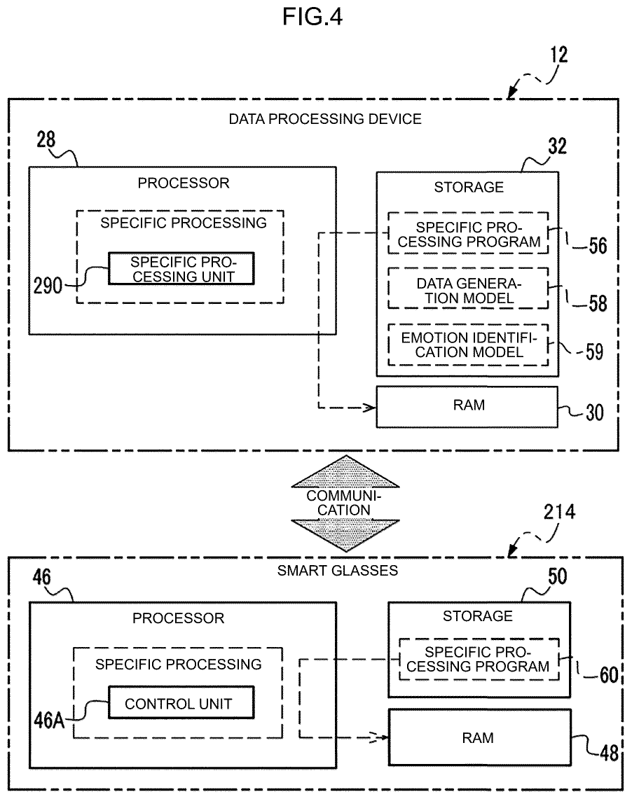

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned operation detection unit, analysis unit, notification unit, fall detection unit, instruction unit, and health monitoring unit is implemented by at least one of, for example, smart glasses 214 and a data processing apparatus 12. For example, the operation detection unit detects the movement of an elderly person using a motion sensor of the smart glasses 214. The analysis unit is implemented, for example, by a specific processing unit 290 of the data processing apparatus 12 and analyzes operation data. The notification unit is implemented, for example, by a control unit 46A of the smart glasses 214 and notifies family members or caregivers when an abnormality is detected. The fall detection unit detects a fall of the elderly person using, for example, an acceleration sensor of the smart glasses 214. The instruction unit is implemented, for example, by the specific processing unit 290 of the data processing apparatus 12 and instructs emergency response. The health monitoring unit monitors the health status of the elderly person using, for example, a medical monitoring system of the smart glasses 214. The correspondence between each unit and the device or control unit is not limited to the examples described above and various modifications are possible.

### Third Embodiment

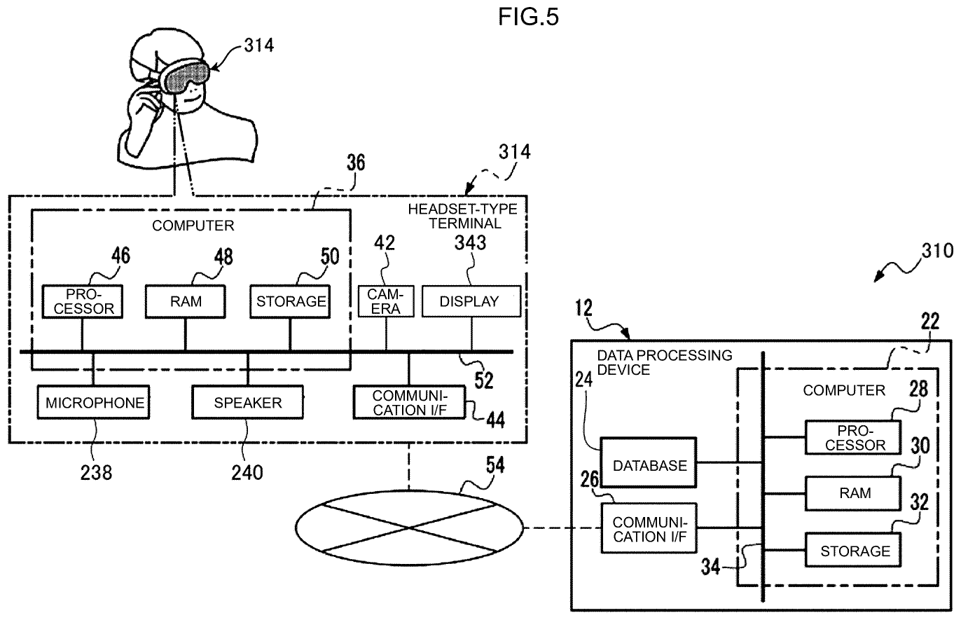

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

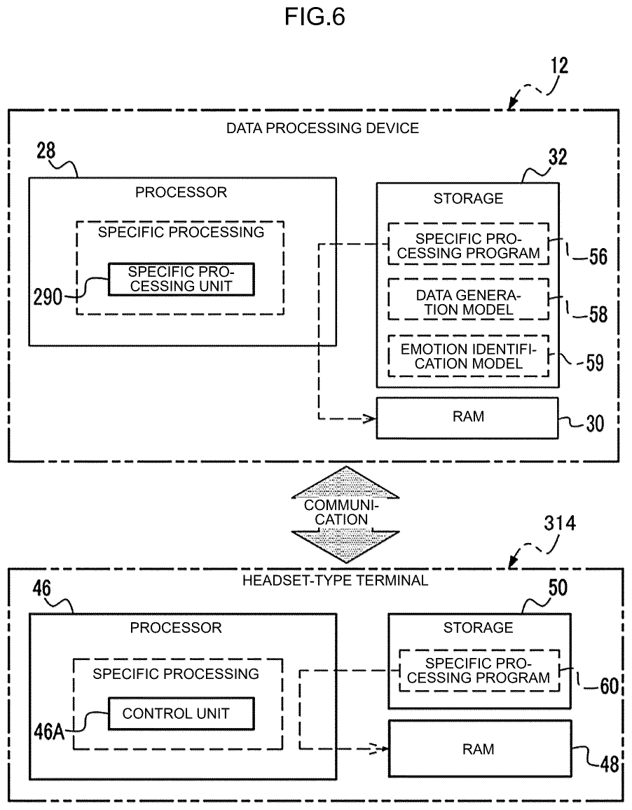

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned operation detection unit, analysis unit, notification unit, fall detection unit, instruction unit, and health monitoring unit is implemented by at least one of, for example, a headset-type terminal 314 and a data processing apparatus 12. For example, the operation detection unit detects the movement of an elderly person using a motion sensor of the headset-type terminal 314. The analysis unit is implemented, for example, by a specific processing unit 290 of the data processing apparatus 12 and analyzes operation data. The notification unit is implemented, for example, by a control unit 46A of the headset-type terminal 314 and notifies family members or caregivers when an abnormality is detected. The fall detection unit detects a fall of the elderly person using, for example, an acceleration sensor of the headset-type terminal 314. The instruction unit is implemented, for example, by the specific processing unit 290 of the data processing apparatus 12 and instructs emergency response. The health monitoring unit monitors the health status of the elderly person using, for example, a medical monitoring system of the headset-type terminal 314. The correspondence between each unit and the device or control unit is not limited to the examples described above and various modifications are possible.

### Fourth Embodiment

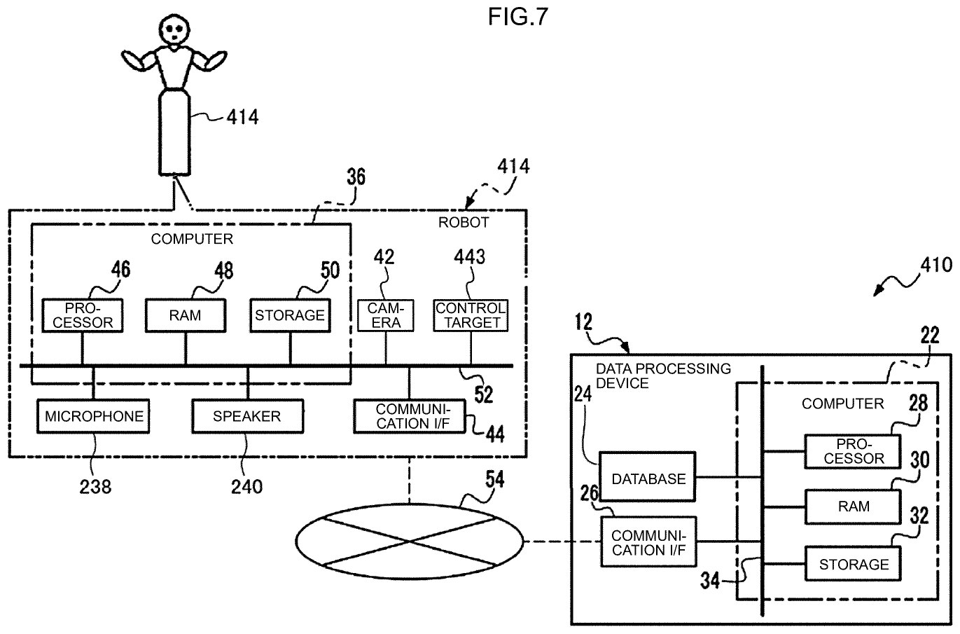

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

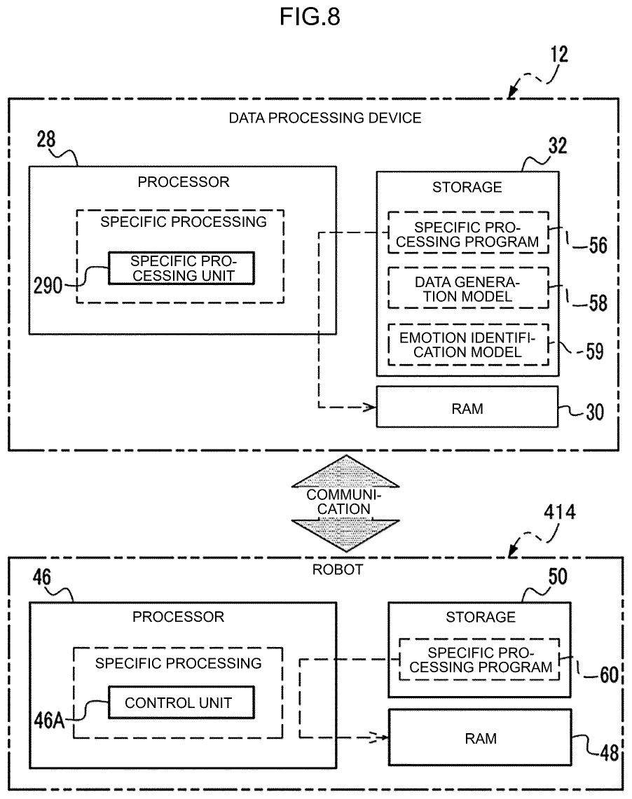

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned operation detection unit, analysis unit, notification unit, fall detection unit, instruction unit, and health monitoring unit is implemented by at least one of, for example, a robot 414 and a data processing apparatus 12. For example, the operation detection unit detects the movement of an elderly person using a motion sensor of the robot 414. The analysis unit is implemented, for example, by a specific processing unit 290 of the data processing apparatus 12 and analyzes operation data. The notification unit is implemented, for example, by a control unit 46A of the robot 414 and notifies family members or caregivers when an abnormality is detected. The fall detection unit detects a fall of the elderly person using, for example, an acceleration sensor of the robot 414. The instruction unit is implemented, for example, by the specific processing unit 290 of the data processing apparatus 12 and instructs emergency response. The health monitoring unit monitors the health status of the elderly person using, for example, a medical monitoring system of the robot 414. The correspondence between each unit and the device or control unit is not limited to the examples described above and various modifications are possible.

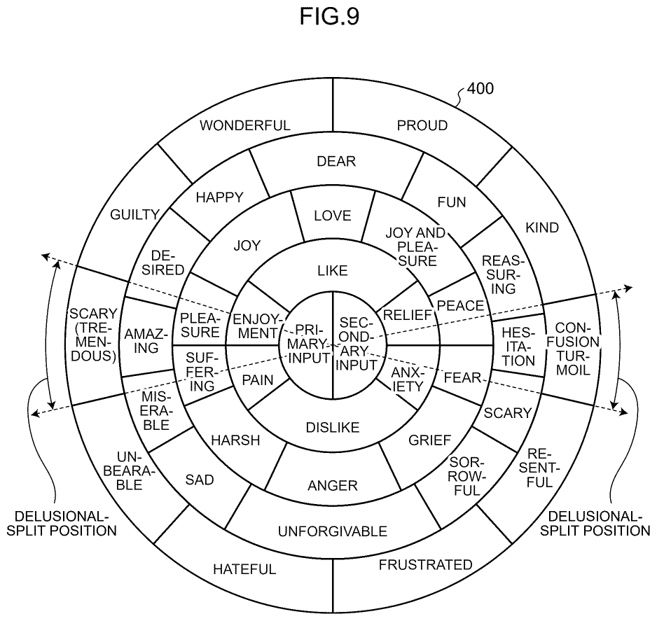

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

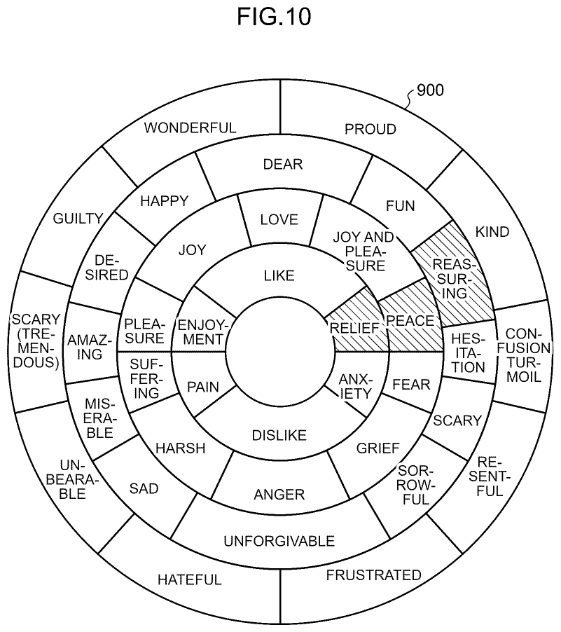

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used. The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1) A system comprising: an operation detection unit configured to detect an operation; an analysis unit configured to analyze information detected by the operation detection unit; a notification unit configured to provide a notification based on information analyzed by the analysis unit; a fall detection unit configured to detect a fall; the analysis unit configured to analyze information detected by the fall detection unit; an instruction unit configured to instruct emergency response based on information analyzed by the analysis unit; a health monitoring unit configured to monitor health; the analysis unit configured to analyze information monitored by the health monitoring unit; and the notification unit configured to provide a notification based on information analyzed by the analysis unit.
(Supplementary Note 2) The system according to Supplementary Note 1, wherein the operation detection unit is installed in various locations in a room and monitors the movement of an elderly person in real time.
(Supplementary Note 3) The system according to Supplementary Note 1, wherein the fall detection unit is worn on the body of an elderly person and immediately detects when a fall occurs.
(Supplementary Note 4) The system according to Supplementary Note 1, wherein the health monitoring unit measures the body temperature, heart rate, and blood pressure of an elderly person in real time.
(Supplementary Note 5) The system according to Supplementary Note 1, wherein the analysis unit analyzes information from the operation detection unit, the fall detection unit, and the health monitoring unit, and notifies family members or caregivers when an abnormality is detected.
(Supplementary Note 6) The system according to Supplementary Note 1, wherein the notification unit notifies family members or caregivers based on information analyzed by AI.
(Supplementary Note 7) The system according to Supplementary Note 1, wherein the instruction unit instructs emergency response based on information analyzed by AI.
(Supplementary Note 8) The system according to Supplementary Note 1, wherein the notification unit has a function of periodically reporting the status of the elderly person to family members or caregivers by AI.
(Supplementary Note 9) The system according to Supplementary Note 1, wherein the notification unit has a function that enables direct communication between family members or caregivers and the elderly person in an emergency.
(Supplementary Note 10) The system according to Supplementary Note 1, wherein the operation detection unit estimates the emotion of the elderly person and adjusts the sensitivity of operation detection based on the estimated emotion.
(Supplementary Note 11) The system according to Supplementary Note 1, wherein the operation detection unit analyzes past operation patterns of the elderly person and improves the accuracy of abnormal operation detection.
(Supplementary Note 12) The system according to Supplementary Note 1, wherein the operation detection unit optimizes detection accuracy based on environmental conditions in the room during operation detection.
(Supplementary Note 13) The system according to Supplementary Note 1, wherein the operation detection unit estimates the emotion of the elderly person and determines the priority of operation detection based on the estimated emotion.
(Supplementary Note 14) The system according to Supplementary Note 1, wherein the operation detection unit adjusts the detection range based on the arrangement of furniture in the room during operation detection.
(Supplementary Note 15) The system according to Supplementary Note 1, wherein the operation detection unit filters the movement of pets during operation detection to prevent false detection.
(Supplementary Note 16) The system according to Supplementary Note 1, wherein the analysis unit estimates the emotion of the elderly person and adjusts the analysis algorithm based on the estimated emotion.
(Supplementary Note 17) The system according to Supplementary Note 1, wherein the analysis unit refers to past abnormal detection data during analysis to improve analysis accuracy.
(Supplementary Note 18) The system according to Supplementary Note 1, wherein the analysis unit adjusts the level of detail of analysis according to the frequency of abnormal detection during analysis.
(Supplementary Note 19) The system according to Supplementary Note 1, wherein the analysis unit estimates the emotion of the elderly person and adjusts the display method of analysis results based on the estimated emotion.
(Supplementary Note 20) The system according to Supplementary Note 1, wherein the analysis unit applies different analysis algorithms according to the type of abnormal detection during analysis.
(Supplementary Note 21) The system according to Supplementary Note 1, wherein the analysis unit determines the priority of analysis based on the time zone of abnormal detection during analysis.
(Supplementary Note 22) The system according to Supplementary Note 1, wherein the notification unit estimates the emotion of the elderly person and adjusts the content of the notification based on the estimated emotion.
(Supplementary Note 23) The system according to Supplementary Note 1, wherein the notification unit refers to the past response history of family members or caregivers during notification to optimize the notification method.
(Supplementary Note 24) The system according to Supplementary Note 1, wherein the notification unit adjusts the urgency of notification according to the type of abnormality during notification.
(Supplementary Note 25) The system according to Supplementary Note 1, wherein the notification unit estimates the emotion of the elderly person and adjusts the timing of notification based on the estimated emotion.
(Supplementary Note 26) The system according to Supplementary Note 1, wherein the notification unit selects the optimal notification method by considering the geographic location information of family members or caregivers during notification.
(Supplementary Note 27) The system according to Supplementary Note 1, wherein the notification unit adjusts the format of notification by considering the device information of family members or caregivers during notification.
(Supplementary Note 28) The system according to Supplementary Note 1, wherein the fall detection unit estimates the emotion of the elderly person and adjusts the sensitivity of fall detection based on the estimated emotion.
(Supplementary Note 29) The system according to Supplementary Note 1, wherein the fall detection unit analyzes past fall history of the elderly person and improves the accuracy of fall detection.
(Supplementary Note 30) The system according to Supplementary Note 1, wherein the fall detection unit optimizes detection accuracy based on the physical condition and clothing of the elderly person during fall detection.
(Supplementary Note 31) The system according to Supplementary Note 1, wherein the fall detection unit estimates the emotion of the elderly person and determines the priority of fall detection based on the estimated emotion.
(Supplementary Note 32) The system according to Supplementary Note 1, wherein the fall detection unit adjusts detection accuracy based on the material and condition of the floor during fall detection.
(Supplementary Note 33) The system according to Supplementary Note 1, wherein the fall detection unit instructs different responses according to the impact level of the fall during fall detection.
(Supplementary Note 34) The system according to Supplementary Note 1, wherein the instruction unit estimates the emotion of the elderly person and adjusts the content of emergency response instructions based on the estimated emotion.
(Supplementary Note 35) The system according to Supplementary Note 1, wherein the instruction unit refers to past response history during emergency response to select the optimal response method.
(Supplementary Note 36) The system according to Supplementary Note 1, wherein the instruction unit adjusts the level of detail of response according to the type of abnormality during emergency response.
(Supplementary Note 37) The system according to Supplementary Note 1, wherein the instruction unit estimates the emotion of the elderly person and determines the priority of emergency response based on the estimated emotion.
(Supplementary Note 38) The system according to Supplementary Note 1, wherein the instruction unit selects the optimal response method by considering the geographic location information of family members or caregivers during emergency response.
(Supplementary Note 39) The system according to Supplementary Note 1, wherein the instruction unit adjusts the format of response by considering the device information of family members or caregivers during emergency response.
(Supplementary Note 40) The system according to Supplementary Note 1, wherein the health monitoring unit estimates the emotion of the elderly person and adjusts the sensitivity of health monitoring based on the estimated emotion.
(Supplementary Note 41) The system according to Supplementary Note 1, wherein the health monitoring unit analyzes past health data of the elderly person and improves the accuracy of abnormality detection.
(Supplementary Note 42) The system according to Supplementary Note 1, wherein the health monitoring unit optimizes monitoring accuracy based on seasonal and weather influences during health monitoring.
(Supplementary Note 43) The system according to Supplementary Note 1, wherein the health monitoring unit estimates the emotion of the elderly person and determines the priority of health monitoring based on the estimated emotion.
(Supplementary Note 44) The system according to Supplementary Note 1, wherein the health monitoring unit improves the accuracy of abnormality detection based on health data of family members or caregivers during health monitoring.
(Supplementary Note 45) The system according to Supplementary Note 1, wherein the health monitoring unit applies different monitoring algorithms according to the type of abnormality during health monitoring.

What is claimed is:

1. A system comprising:
circuitry configured to:
receive, from a client terminal via a communication interface and a packet-switched network, sensor data comprising at least one of acceleration data from a three-axis acceleration sensor, infrared sensor data, or biometric data;
extract, using a machine learning model comprising at least one of a convolutional neural network or a recurrent neural network, a feature vector from the sensor data;
compute an anomaly score by comparing the feature vector with reference data;
detect an anomaly based on the anomaly score exceeding a threshold;
generate, using a data generation model obtained by deep learning on a neural network, inference data comprising at least one of an alert classification label or a response instruction; and
transmit the inference data to the client terminal via the communication interface and the packet-switched network, the inference data causing the client terminal to output the inference data to a user.

2. The system according to claim 1, wherein the sensor data comprises motion sensor data obtained from a plurality of motion sensors installed in different locations, and wherein the circuitry is configured to monitor movement of the user in real time by acquiring position coordinates and movement status as three-dimensional vector data.

3. The system according to claim 1, wherein the sensor data comprises acceleration data sampled at 100 Hz or higher from an acceleration sensor worn on a body of the user, and wherein the circuitry is configured to detect a sudden change in acceleration pattern indicating an anomalous movement event.

4. The system according to claim 1, wherein the biometric data comprises at least one of body temperature data, heart rate data, or blood pressure data, and wherein the circuitry is configured to detect a vital sign anomaly based on the biometric data.

5. The system according to claim 1, wherein the circuitry is further configured to preprocess the sensor data by performing at least one of noise removal using a filter, outlier removal, or normalization before extracting the feature vector.

6. The system according to claim 1, wherein the machine learning model comprises at least one of a long short-term memory network or a gated recurrent unit, and wherein the circuitry is configured to analyze time-series patterns in the sensor data.

7. The system according to claim 1, wherein the circuitry is further configured to estimate an emotion of the user by applying an emotion identification model to the sensor data, and to adjust the threshold for detecting the anomaly based on the estimated emotion.

8. The system according to claim 7, wherein the circuitry is configured to lower the threshold when the estimated emotion indicates anxiety and to raise the threshold when the estimated emotion indicates relaxation.

9. The system according to claim 1, wherein the circuitry is further configured to store past sensor data in a database, analyze past anomaly patterns associated with the user, and adjust the threshold based on the analyzed past anomaly patterns.

10. The system according to claim 1, wherein the circuitry is further configured to receive environmental sensor data comprising at least one of temperature data, humidity data, or illuminance data, and to adjust the threshold based on the environmental sensor data.

11. The system according to claim 1, wherein the circuitry is further configured to determine a priority of processing the sensor data based on a time of day, such that sensor data received during a nighttime period is processed with a higher priority than sensor data received during a daytime period.

12. The system according to claim 1, wherein the circuitry is further configured to classify a type of the anomaly as one of a plurality of anomaly categories comprising a movement anomaly, a vital sign anomaly, or a combined anomaly, and to generate the inference data based on the classified anomaly type.

13. The system according to claim 1, wherein the circuitry is further configured to compute an impact level score based on a magnitude of the anomaly, and to generate the response instruction based on the impact level score, such that a high impact level score triggers an emergency response instruction and a low impact level score triggers an observation instruction.

14. The system according to claim 1, wherein the circuitry is further configured to receive geographic location information from the client terminal, and to select a notification recipient based on a distance between the geographic location information and a location of the notification recipient.

15. The system according to claim 1, wherein the circuitry is further configured to select a notification method from a plurality of notification methods comprising a push notification, an SMS message, an email, or a voice call, based on device information of a notification recipient.

16. The system according to claim 1, wherein the circuitry is further configured to generate a periodic status report by aggregating the sensor data over a predetermined time period and transmit the periodic status report to a registered recipient via the communication interface.

17. The system according to claim 1, wherein the circuitry is further configured to filter movement patterns associated with a non-human entity by applying an object detection algorithm to image data received from the client terminal, and to exclude the filtered movement patterns from the anomaly detection.

18. A system comprising:
a communication interface configured to communicate, via a packet-switched network conforming to at least one of a 5G, Wi-Fi, or Bluetooth communication standard, with a client terminal comprising a three-axis acceleration sensor, an infrared sensor, a microphone, a speaker, and a display;
a processor;
a random-access memory;
a memory storing a data generation model obtained by deep learning on a neural network, and an emotion identification model;
a database; and
circuitry configured to:
receive, from the client terminal via the communication interface, sensor data comprising at least one of acceleration data from the three-axis acceleration sensor, infrared sensor data from the infrared sensor, or biometric data;
preprocess the sensor data by performing at least one of noise removal, outlier removal, or normalization;
extract, using a machine learning model comprising at least one of a convolutional neural network, a recurrent neural network, or a long short-term memory network, a feature vector from the preprocessed sensor data;
compute an anomaly score by comparing the feature vector with reference data stored in the database;
detect an anomaly based on the anomaly score exceeding a threshold;
estimate an emotion of the user by applying the emotion identification model to the sensor data;
adjust the threshold based on the estimated emotion;
generate, using the data generation model, inference data comprising at least one of an alert classification label, a response instruction, or a status report, the inference data being adapted based on the estimated emotion; and
transmit the inference data to the client terminal via the communication interface, the inference data causing the client terminal to output the inference data to the user via at least one of the display or the speaker.

19. The system according to claim 18, wherein the data generation model comprises at least one of a text generation AI, an image generation AI, or a multimodal generation AI, and wherein the data generation model is a fine-tuned model configured to output inference results from prompts without instructions.

20. A method performed by circuitry of a system comprising a communication interface, a memory storing a data generation model obtained by deep learning on a neural network, and a database, the method comprising:
receiving, from a client terminal via the communication interface and a packet-switched network, sensor data comprising at least one of acceleration data from a three-axis acceleration sensor, infrared sensor data, or biometric data;
extracting, using a machine learning model comprising at least one of a convolutional neural network or a recurrent neural network, a feature vector from the sensor data;
computing an anomaly score by comparing the feature vector with reference data stored in the database;
detecting an anomaly based on the anomaly score exceeding a threshold;
generating, using the data generation model, inference data comprising at least one of an alert classification label or a response instruction; and
transmitting the inference data to the client terminal via the communication interface and the packet-switched network, the inference data causing the client terminal to output the inference data to a user.
